(19) United States
(12) Patent Application Publication
(10) Pub. No.: US 2011/0251524 A1
(43) Pub. Date: Oct. 13, 2011

(54) DEVICE FOR ULTRASOUND TREATMENT AND MONITORING TISSUE TREATMENT

(75) Inventors: Haim Azhari, Doar-Na Misgav (IL); Liat Tsoref, Tel Aviv (IL); Yossi Gross, Moshav Mazor (IL); Jacob Benarie, Haifa (IL)

(73) Assignee: SLENDER MEDICAL, LTD., Herzliya (IL)

(21) Appl. No.: 13/063,567

(22) PCT Filed: Sep. 13, 2009
(86) PCT No.: PCT/IL09/00894

§ 371 Date: May 5, 2011

Related U.S. Application Data
(63) Continuation-in-part of application No. 12/207,043, filed on Sep. 9, 2008.
Application No. 12/207,043 is a continuation-in-part of application No. 11/651,198, filed on Jan. 8, 2007, now Pat. No. 7,828,734.
Application No. PCT/IL09/00894 is a continuation-in-part of application No. 11/653,115, filed on Jan. 12, 2007.
(60) Provisional application No. 61/096,419, filed on Sep. 12, 2008.
(60) Provisional application No. 60/780,772, filed on Mar. 9, 2006.
(60) Provisional application No. 60/809,577, filed on May 30, 2006.
(60) Provisional application No. 60/860,635, filed on Nov. 22, 2006.

(51) Int. Cl. A61N 7/00

(52) U.S. Cl. 601/2

(57) ABSTRACT

Apparatus is provided, including first and second support structures for placement on skin of a subject. At least one of the first and second structures is moveable with respect to the other so as to draw a portion of the skin and underlying tissue of the subject between respective lateral surfaces of the support structures. At least one ultrasound transducer is moveably coupled to the first support structure along an axis of the structure that is not parallel to the lateral surface of the structure. The ultrasound transducer is configured to transmit toward the portion of skin and underlying tissue one or more forms of acoustic radiation energy, including treatment energy. At least one acoustic element is coupled to the second support structure. Other applications are also described.

### CROSS-REFERENCES TO RELATED APPLICATIONS

The present application:

(a) claims priority to U.S. Provisional Patent Application 61/096,419 to Azhari et al., entitled, “A device for ultrasound treatment and monitoring tissue treatment,” filed Sep. 12, 2008; and

(b) is a continuation-in-part of U.S. patent application Ser. No. 12/207,043 to Azhari et al, entitled, “A device for ultrasound monitored tissue treatment,” filed Sep. 9, 2008, which is a national phase application of PCT/IL2007/000307, which:
(a) claims the priority of:
   U.S. Provisional Patent Application 60/780,772 to Azhari et al., filed Mar. 9, 2006, entitled, “A method and system for lypolysis and body contouring,”
   U.S. Provisional Patent Application 60/809,577 to Azhari et al., filed May 30, 2006, entitled, “A device for ultrasound monitored tissue treatment,” and
   U.S. Provisional Patent Application 60/860,635 to Azhari et al., filed Nov. 22, 2006, entitled, “Cosmetic tissue treatment using ultrasound,” and
(b) is a continuation-in-part of and claims the priority of:
   U.S. patent application Ser. No. 11/651,198 to Azhari et al. filed Jan. 8, 2007, entitled, “A device for ultrasound monitored tissue treatment,” and
   U.S. patent application Ser. No. 11/653,115 to Azhari et al., filed Jan. 12, 2007, entitled, “A method and system for lipolysis and body contouring.”

The present application is related to U.S. Provisional Patent Application 61/096,516 to Azhari, entitled, “Virtual ultrasonic scissors—A non-invasive method for tissue treatment,” filed Sep. 12, 2008, and to a PCT application to Azhari, entitled “Virtual ultrasonic scissors,” filed on even date herewith that claims priority of the '516 Azhari provisional application.

All of these applications are incorporated herein by reference.

### FIELD OF THE INVENTION

Some applications of the present invention relate in general to tissue treatment by application of energy thereto, and specifically to the monitoring and applying of ultrasound to skin and underlying tissue.

### BACKGROUND OF THE INVENTION

Systems for applying energy to biological tissue are well known. Such energy application may be intended to heal injured tissue, ablate tissue, or improve the appearance of tissue. Energy may be applied in different forms, such as radiofrequency, laser, or ultrasound.

The following references may be of interest:

US Patent Application Publications 2002/0193831, 2003/0083536, 2004/0039312, 2004/0215110, 2004/0217675, 2005/0049543, 2005/0154308, 2005/0154309, 2005/0193451, 2005/0154295, 2005/0154313, 2005/0154314, 2005/0154431, 2005/0187463, 2005/0187495, 2005/0261584, 2006/0058707, 2006/0036300, 2006/0094988, and 2006/0122509; U.S. Pat. Nos. 4,355,643, 5,143,063, 5,573,497, 5,575,772, 5,601,526, 5,665,053, 5,743,863, 6,113,558, 6,350,245, 6,438,424, 6,450,979, 6,500,141, 6,508,813, 6,607,498, 6,626,854, 6,645,162, 6,730,034, 6,758,845, 6,971,994, and 7,258,674; and PCT Patent Publications WO 00/053263, WO 01/92846, WO 05/065371, WO 05/065409, WO 05/074365, WO 06/018837, WO 06/080012, and WO 07/102,161.

Akashi N et al., “Acoustic properties of selected bovine tissue in the frequency range 20-200 MHz,” J Acoust Soc Am. 98 (6):3035-9 (1995)

Bommannanl D et al., “Sonophoresis. II. Examination of the Mechanism(s) of Ultrasound-Enhanced Transdermal Drug Delivery,” Journal Pharmaceutical Research, 9 (8):1043-47 (1992)

Laubach H J et al., “Intense focused ultrasound: evaluation of a new treatment modality for precise microcoagulation within the skin,” Dermatol Surg 34:727-734 (2008)

Levy D et al., “Effect of Ultrasound on Transdermal Drug Delivery to Rats and Guinea Pigs” J. Clin. Invest. Volume 83, pp. 2074-2078 (1989)

Moran C M et al., “Ultrasonic propagation properties of excised human skin,” Ultrasound Med Biol. 21 (9):1177-90 (1995)

### SUMMARY OF THE INVENTION

In some applications of the invention, cosmetic and/or medical apparatus is provided which comprises a tissue monitoring system and a tissue treatment system. The monitoring system assesses a state of tissue of a subject, and the treatment system applies a treatment to the tissue. The treatment typically includes various cosmetic treatments (e.g., body contouring by lipolysis, hair removal, treatment of eye bags, treatment of lipomas, wrinkle and face lift, or face-localized molding of adipose tissue). Typically, the monitoring and treatment occur in alternation, until the monitoring system determines that the treatment has been completed. For some applications, the treatment and monitoring systems are coupled to a housing, and the tissue (e.g., skin and underlying tissue) of the subject is sucked at least partially into the housing, to allow the system to monitor or treat (as appropriate) the tissue that has been sucked into the housing. In this case, the system typically transmits ultrasound energy that is designated to remain in large part within the housing and tissue therein, and generally not to affect tissue outside of the housing.

As appropriate for a given application, the system comprising the housing may be the monitoring system, the treatment system, or both the monitoring system and the treatment system.

In some applications of the present invention, two support structures, or holders, are configured to be placed perpendicularly with respect to the surface of the skin of the subject. Typically, at least one of the support structures is movable or rotatable with respect to the second support structure in order to draw, pinch, or clamp a portion of the skin and underlying tissue between the two support structures. At first acoustic element is coupled to the first support structure, and a second acoustic element is coupled to the second support structure in a manner in which the first acoustic element is disposed opposite and facing the second acoustic element. This enables through-transmitted energy from the first acoustic element to reach the second acoustic element, and vice versa.

Typically, the first and second acoustic elements are moveably coupled to the first and second support structures, respectively, along respective axes thereof that are not parallel, e.g., perpendicular, to the lateral surfaces of the first and second support structures. These lateral surfaces are configured to be in contact with the portion of skin and underlying tissue that is drawn between the first and second support structures. Thus, the location of the at least one focal zone generated within the portion of skin and underlying tissue by the acoustic element(s) is alterable by sliding one or both of the acoustic elements along the axis while the support structures remain fixed in place.

For some applications of the present invention, one or more acoustic lens are in acoustic communication with one or more of the acoustic elements in order to converge the ultrasound beams transmitted from the acoustic element toward at least one focal zone in the portion of skin and underlying tissue. For some applications of the present invention, the focal point of the ultrasound in the tissue is moved by either (a) moving the lens with respect to the acoustic element while support structures remain in place pinching the portion of tissue, or (b) by changing the curvature of the lens responsively to commands from a control unit.

The acoustic elements are positioned with respect to the housing such that ultrasound energy transmitted by the transducers remains generally within a plane defined by the housing. Similarly, in applications in which the monitoring system comprises the housing, the transducers are typically disposed such that they are optimized to receive ultrasound energy coming generally from within the plane.

There is therefore provided, in accordance with some applications of the present invention, apparatus, including:

a first support structure having a lower surface and a lateral surface, the first support structure being configured for placement on a first location on skin of a subject;

a second support structure having a lower surface and a lateral surface, the second support structure being configured for placement on a second location on the skin of the subject, at least one of the first and second support structures being moveable with respect to the other so as to draw a portion of the skin and underlying tissue of the subject between the respective lateral surfaces of the first and second support structures;

at least one ultrasound transducer moveably coupled to the first support structure, the ultrasound transducer being moveable along an axis of the first support structure that is not parallel to the lateral surface of the first support structure, and configured to transmit toward the portion of skin and underlying tissue one or more forms of acoustic radiation energy, at least one of the one or more forms of energy including treatment energy; and

at least one acoustic element coupled to the second support structure.

In some applications of the present invention, the axis is perpendicular to the lateral surface of the first support structure, and the ultrasound transducer is moveable along the axis that is perpendicular to the lateral surface of the first support structure.

In some applications of the present invention, the ultrasound transducer and the acoustic element are arranged such that transmission of energy from the ultrasound transducer generates a focal zone which moves within the portion of skin and underlying tissue in accordance with movement of the at least one ultrasound transducer.

In some applications of the present invention, the ultrasound transducer includes an ultrasound transducer shaped so as to define a curved surface in acoustic communication with the lateral surface of the first support structure.

In some applications of the present invention, the apparatus is configured to configure the treatment energy to generate a standing wave in the portion of skin and underlying tissue.

In some applications of the present invention, the apparatus is configured to configure the treatment energy to generate constructive interference in the portion of skin and underlying tissue.

In some applications of the present invention, the apparatus is configured to configure the treatment energy to generate an imploding wave in the portion of skin and underlying tissue.

In some applications of the present invention:

at least a portion of the lateral surface of the first support structure is configured to be disposed at a first interface between the portion of skin and the housing,

at least a portion of the lateral surface of the second support structure is configured to be disposed at a second interface between the portion of skin and the housing, the second interface being opposite the first interface, and

the ultrasound transducer and the acoustic element are configured to facilitate confocal transmission of the one or more forms of energy in the portion of skin and underlying tissue, from the opposing interfaces.

In some applications of the present invention, the first and second support structures include first and second pinching elements, respectively, and the first and second pinching elements are configured to facilitate pinching of the portion of skin and underlying tissue.

In some applications of the present invention, the apparatus includes a suction apparatus configured to draw the portion of skin and underlying tissue between the first and second support structures.

In some applications of the present invention, the ultrasound transducer is configured to transmit the energy at an angle that is less than 10 degrees with respect to the lower surface of the first support structure.

In some applications of the present invention, the apparatus includes a processing unit, and the processing unit is configured to detect a position change of the at least one of the support structures in response to movement of the at least one of the support structures, and responsively thereto, to facilitate monitoring of the parameter of the tissue underlying the skin.

In some applications of the present invention:

the first support structure includes a track disposed along the axis of the first support structure, and

the ultrasound transducer is moveably coupled to the track.

In some applications of the present invention, the ultrasound transducer is configured to transmit between 0.5 Watts/cm̂2 and 100 Watts/cm̂2.

In some applications of the present invention, the ultrasound transducer is configured to transmit between 1.5 Watts/cm̂2 and 30 Watts/cm̂2.

In some applications of the present invention, the acoustic element is moveable along an axis of the second support structure that is not parallel to the lateral surface of the second support structure.

In some applications of the present invention, the acoustic element is moveable along an axis of the second support structure that is perpendicular to the lateral surface of the second support structure.

In some applications of the present invention:

the second support includes a track disposed along the axis of the second support structure, and

the acoustic element is moveably coupled to the track.

In some applications of the present invention:

the at least one ultrasound transducer defines a first ultrasound transducer configured to transmit energy toward the portion of skin and underlying tissue in a manner in which a first focal zone is created in the portion of skin and underlying tissue, and

the first transducer is configured to alter a position of the first focal zone by being moved along the axis.

In some applications of the present invention, the first ultrasound transducer is configured to transmit the energy in a manner in which the first focal zone is generated in the portion of skin and the underlying tissue substantially midway between the first ultrasound transducer and the acoustic element.

In some applications of the present invention:

the acoustic element is moveable with respect to the second support structure along an axis of the second support structure that is not parallel to the lateral surface of the second support structure,

the acoustic element defines a second ultrasound transducer configured to transmit energy toward the portion of skin and underlying tissue such that a second focal zone is generated in the portion of skin and underlying tissue, and

the second transducer is configured to alter a position of the second focal zone by being moved along the axis of the second support structure.

In some applications of the present invention, the first and second ultrasound transducers are arranged such that transmission of energy from the first and second ultrasound transducers causes the first and second focal zones to overlap at least in part.

In some applications of the present invention, the second ultrasound transducer is configured to transmit the energy in a manner in which the second focal zone is generated in the portion of skin and the underlying tissue substantially midway between the first and second ultrasound transducers.

In some applications of the present invention, the acoustic element includes an acoustic reflector configured to reflect the energy transmitted from the first ultrasound transducer toward at least the first focal zone.

In some applications of the present invention:

the apparatus further includes a first acoustic lens configured to be disposed between the ultrasound transducer and the portion of skin and the underlying tissue within the at least a part of the housing, and

the first lens is moveable with respect to the first support structure along the axis of the first support structure that is not parallel to the lateral surface of the first support structure.

In some applications of the present invention, the first lens is configured to alter a position of the first focal zone by being moved along the axis of the first support structure.

In some applications of the present invention, the first lens is configured to contact the skin and to be easily removable, by a user of the apparatus, from other components of the apparatus.

In some applications of the present invention, the acoustic lens is shaped to provide a first lateral surface and a second lateral surface having two or more concave surfaces, and the acoustic lens is configured to be disposed between the ultrasound transducer and a first surface of the portion of skin and underlying tissue in a manner in which:

the first lateral surface of the acoustic lens is in acoustic communication with the ultrasound transducer, and

the second lateral surface of the acoustic lens is in acoustic communication with the lateral surface of the first support structure and with the first surface of the portion of skin and underlying tissue.

In some applications of the present invention, the first lateral surface of the acoustic lens is planar.

In some applications of the present invention, the second lateral surface of the acoustic lens includes a plurality of concave surfaces, and each concave surface is configured to focus the energy transmitted from the ultrasound transducer toward a respective focal zone in the first surface of the portion of skin and underlying tissue.

In some applications of the present invention, the at least one acoustic lens is moveable with respect to the first support structure along the axis that is not parallel to the lateral surface of the first support structure.

In some applications of the present invention, each one of the two or more concave surfaces of the second lateral surface of the acoustic lens is configured to focus the energy toward a respective focal zone in the first surface of the portion of skin and underlying tissue.

In some applications of the present invention, each one of the two or more concave surfaces of the second lateral surface of the acoustic lens is configured to facilitate generating of a respective hole at each focal zone in the first surface of the portion of skin and underlying tissue.

In some applications of the present invention:

the apparatus further includes a second acoustic lens configured to be disposed between the second acoustic element and the portion of skin and the underlying tissue within the at least a part of the housing,

the second lens is moveable with respect to the second support structure along an axis of the second support structure that is not parallel to the lateral surface of the second support structure, and

the second lens is configured to focus the transmitted energy to at least one second focal zone.

In some applications of the present invention, the second lens is moveable with respect to the second support structure along an axis of the second support structure that is perpendicular to the lateral surface of the second support structure.

In some applications of the present invention, the second lens is configured to alter a position of the second focal zone by being moved along the axis of the second support structure.

In some applications of the present invention, the first and second lenses are configured to focus the transmitted energy toward the portion of skin and underlying tissue in a manner in which the first and second focal zones overlap at least in part.

In some applications of the present invention, the second lens is configured to contact the skin and to be easily removable, by a user of the apparatus, from other components of the apparatus.

In some applications of the present invention, the second support structure is configured to be disposed with respect to the ultrasound transducer such that the one or more forms of energy are through-transmitted toward the acoustic element, and at least a portion of the through-transmitted energy is received at least one component selected from the group consisting of the at least one ultrasound transducer and the at least one acoustic element.

In some applications of the present invention, the ultrasound transducer is configured to transmit through the portion of skin and underlying tissue one or more forms of acoustic radiation energy, at least one of the one or more forms of energy including treatment energy, and the ultrasound transducer and the acoustic element are configured to facilitate confocal transmission of the form of energy.

In some applications of the present invention, the apparatus includes a processing unit configured to monitor a change in a parameter of the tissue underlying the skin, responsively to the received energy.

In some applications of the present invention:

the ultrasound transducer includes a first ultrasound transducer,

the acoustic element includes a second ultrasound transducer configured to transmit one or more forms of energy through the tissue, the one or more forms of energy including treatment energy,

the first ultrasound transducer is configured to receive at least a portion of the one or more forms of energy transmitted from the second ultrasound transducer, and

the processing unit is configured to monitor a change in the parameter of the tissue responsively to the energy received by the first ultrasound transducer from the second ultrasound transducer.

In some applications of the present invention:

the acoustic element includes an ultrasound reflector configured to reflect the through-transmitted energy transmitted from the ultrasound transducer,

the ultrasound transducer is configured to receive at least a portion of the reflected energy, and

the processing unit is configured to monitor a change in the parameter of the tissue responsively to the reflected energy.

In some applications of the present invention, the processing unit is configured to detect adipose tissue in the portion of skin and the underlying tissue.

In some applications of the present invention, the ultrasound transducer includes a plurality of ultrasound transducers.

In some applications of the present invention, the plurality of ultrasound transducers include a phased array of ultrasound transducers, and the phased array of ultrasound transducers is configured to steer a focal zone of energy transmitted within the portion of skin and underlying tissue.

In some applications of the present invention, the plurality of ultrasound transducers is disposed with respect to the first support structure so as to define a portion of at least one or more shapes selected from the group consisting of: a ring and an ellipse.

In some applications of the present invention, the ultrasound transducer and the acoustic element are configured to operate in generally closed looped operation.

In some applications of the present invention, the apparatus includes a processing unit in communication with the ultrasound transducer and the acoustic element, and the processing unit is configured to monitor the alteration of the parameter and regulate the transmission of the treatment energy in response to the monitoring.

There is additionally provided, in accordance with some applications of the present invention, a method, including:

placing at a first location on skin of a subject at least one ultrasound transducer moveably coupled to a first support structure, the ultrasound transducer being configured to transmit through the skin one or more forms of acoustic radiation, at least one of the one or more forms of energy including treatment energy,

placing at a second location on the skin of the subject at least one acoustic element moveably coupled to a second support structure;

drawing at least a portion of the skin and underlying tissue between the at least one ultrasound transducer and the at least one acoustic element;

moving the at least one ultrasound transducer with respect to the first support structure along an axis that is not parallel to a lateral surface of the first support structure;

transmitting from the at least one ultrasound transducer one or more forms of energy through the portion of skin and the underlying tissue; and

generating confocal transmission responsively to the transmitting.

There is further provided, in accordance with some applications of the present invention, a method, including:

placing in communication with a surface of a portion of skin of a subject:
at least one acoustic element, and
at least one acoustic lens between the acoustic element and the surface of the portion of the skin of the subject, the acoustic lens having a first lateral surface that is disposed in acoustic communication with the acoustic element, and a second lateral surface having two or more concave interfaces in acoustic communication with the surface of the portion of the skin of the subject;

transmitting from the at least one acoustic element one or more forms of acoustic radiation energy, at least one of the one or more forms of energy including treatment energy, toward the surface of the portion of the skin of the subject;

focusing the energy through each of the two or more concave interfaces to a respective focal zone at the surface of the portion of the skin of the subject; and

responsively to the focusing, generating two or more treatment areas in the surface of the portion of the skin of the subject.

There is also provided, in accordance with some applications of the present invention, apparatus, including:

a support structure having a lower surface and a lateral surface configured for placement against a surface of a portion of skin of a subject;

at least one acoustic element coupled to the support structure, the acoustic element being configured to transmit one or more forms of acoustic radiation energy, at least one of the one or more forms of energy including treatment energy, toward the surface of the portion of the skin of the subject; and

at least one acoustic lens coupled to the support structure, the at acoustic lens having:
a first lateral surface in communication with the acoustic element, and
a second lateral surfaces having two or more concave interfaces in acoustic communication with the lateral surface of the support structure and configured to be in acoustic communication with the surface of the portion of the skin of the subject,

and each one of the two or more concave interfaces being configured to focus energy transmitted from the at least one acoustic element toward respective focal zones at the surface of the portion of the skin of the subject, and, responsively to generate a respective treatment zone at each focal zone.

In some applications of the present invention, the first lateral surface of the lens is planar.

In some applications of the present invention, each one of the two or more concave interfaces is configured to focus energy transmitted from the at least one acoustic element toward respective focal zones at the surface of the portion of the skin of the subject, and, responsively to generate a respective hole at each focal zone.

In some applications of the present invention, the second lateral surface of the acoustic lens includes a plurality of concave surfaces, and each concave surface is configured to focus the energy transmitted from the ultrasound transducer toward a respective focal zone in the surface of the portion of skin of the subject.

In some applications of the present invention, the apparatus is configured to facilitate uptake by the surface of the portion of the skin of the patient one or more substances selected from the group consisting of: a medication, a solution, or a cream responsively to the generating of the respective holes.

In some applications of the present invention, the at least one acoustic lens is moveable with respect to the support structure along an axis that is not parallel to the lateral surface of the support structure.

In some applications of the present invention, the at least one acoustic lens is moveable with respect to the support structure along an axis that is perpendicular to the lateral surface of the support structure.

In some applications of the present invention, the at least one acoustic lens is configured to alter a respective position of each one of the focal zones in accordance with the movement of the acoustic lens.

In some applications of the present invention, the at least one acoustic element is moveable with respect to the support structure along an axis that is not parallel to the lateral surface of the support structure.

In some applications of the present invention, the at least one acoustic element is moveable with respect to the support structure along an axis that is perpendicular to the lateral surface of the support structure.

In some applications of the present invention, the at least one acoustic element is configured to alter a respective position of each one of the focal zones in accordance with the movement of the acoustic element.

In some applications of the present invention, each one of the two or more concave surfaces of the second lateral surface of the acoustic lens is configured to focus the energy toward a respective focal zone in the surface of the portion of the skin of the subject.

In some applications of the present invention, each one of the two or more concave surfaces of the second lateral surface of the acoustic lens is configured to facilitate generating of a respective hole at each focal zone in the surface of the portion of the skin of the subject.

The following is a list of some of the inventive concepts, in accordance with some embodiments of the present invention, described in this application:

1. Apparatus, comprising:

a housing configured for placement on skin of a subject, and to draw at least a portion of the skin and underlying tissue within at least a part of the housing;

at least one ultrasound transducer coupled to the housing and configured to transmit through the portion of skin and the underlying tissue one or more forms of acoustic radiation energy, at least one of the one or more forms of energy including treatment energy;

at least one acoustic element coupled to the housing and disposed with respect to the ultrasound transducer such that the one or more forms of energy are through-transmitted toward the acoustic element, at least a portion of the through-transmitted energy being received at least one component selected from the group consisting of: the at least one ultrasound transducer and the at least one acoustic element, the acoustic element and the ultrasound transducer being configured to facilitate confocal transmission of the one or more forms of energy in the portion of skin and underlying tissue within the at least a part of the housing; and

a processing unit, configured to monitor the portion of skin and underlying tissue in response to the received energy,

wherein the at least one ultrasound transducer is configured to apply a treatment to the tissue in response to the monitored state of the tissue.

2. The apparatus according to inventive concept 1, wherein

the at least one ultrasound transducer is configured to be disposed at a first interface between the portion of skin and underlying tissue and the housing,

the at least one acoustic element is configured to be disposed at a second interface between the portion of skin and underlying tissue and the housing, the second interface being opposite the first interface, and

the ultrasound transducer and the acoustic element are configured to facilitate confocal transmission of the one or more forms of energy in the portion of skin and underlying tissue, from the opposing interfaces.

3. The apparatus according to inventive concept 1, wherein the ultrasound transducer is configured to transmit the energy at an angle that is less than 10 degrees with respect to a lower surface of the apparatus.
4. The apparatus according to inventive concept 1, wherein the ultrasound transducer comprises an ultrasound transducer shaped so as to define a curved surface configured to be disposed in acoustic communication with portion of the skin and underlying tissue.
5. The apparatus according to inventive concept 1, wherein:

the ultrasound transducer comprises a first ultrasound transducer,

the acoustic element comprises a second ultrasound transducer configured to transmit one or more forms of energy through the tissue, the one or more forms of energy including treatment energy,

the first ultrasound transducer is configured to receive at least a portion of the one or more forms of energy transmitted from the second ultrasound transducer, and

the processing unit is configured to monitor a change in the parameter of the tissue responsively to the energy received by the first ultrasound transducer from the second ultrasound transducer.

6. The apparatus according to inventive concept 1, wherein:

the acoustic element comprises an ultrasound reflector configured to reflect the through-transmitted energy transmitted from the ultrasound transducer,

the ultasound transducer is configured to receive at least a portion of the reflected energy, and

the processing unit is configured to monitor a change in the parameter of the tissue responsively to the reflected energy.

7. The apparatus according to inventive concept 1, wherein the ultrasound transducer comprises a first ultrasound transducer, and wherein the acoustic element comprises a second ultrasound transducer configured to receive at least a portion of the through-transmitted energy from the first ultrasound transducer.
8. The apparatus according to inventive concept 1, wherein the ultrasound transducer is configured to transmit between 0.5 Watts/cm̂2 and 100 Watts/cm̂2.
9. The apparatus according to inventive concept 8, wherein the ultrasound transducer is configured to transmit between 1.5 Watts/cm̂2 and 30 Watts/cm̂2.
10. The apparatus according to inventive concept 1, wherein:

the ultrasound transducer comprises a plurality of ultrasound transducers,

the plurality of ultrasound transducers are disposed in a given relationship with respect to the housing in which at least a first one of the plurality of transducers is disposed opposite at least a second one of the plurality of ultrasound transducers, and

a first portion of the plurality of ultrasound transducers is configured to transmit the energy toward at least one focal zone within a plane defined by the housing.

11. The apparatus according to inventive concept 10, wherein the plurality of ultrasound transducers comprise a phased array of ultrasound transducers, and wherein the phased array of ultrasound transducers is configured to steer a focal zone of energy transmitted within the plane.
12. The apparatus according to inventive concept 10, wherein the plurality of ultrasound transducers is disposed with respect to the housing so as to define a portion of at least one or more shapes selected from the group consisting of: a ring and an ellipse.
13. The apparatus according to inventive concept 10, wherein the first portion of the plurality of ultrasound transducers is configured to transmit the energy in a manner in which the focal zone of the energy is moved over at least a portion of the plane defined by the housing.
14. The apparatus according to inventive concept 1, wherein:

the housing comprises at least first and second support structures configured to be disposed substantially perpendicularly with respect to a surface of skin surrounding the portion of skin and the underlying tissue within at least a part of the housing,

the ultrasound transducer and the acoustic element are coupled to the first and second support structures, respectively, and

at least one of the support structures is movable with respect to the other support structure after the housing comes in contact with the tissue.

15. The apparatus according to inventive concept 14, wherein the first and second support structures comprise first and second pinching elements, respectively, configured to pinch a portion of skin between the first and second support structures.
16. The apparatus according to inventive concept 14, wherein the processing unit is configured to detect a position change of the at least one of the support structures in response to movement of the at least one of the support structures, and responsively thereto, to facilitate monitoring of the parameter of the tissue underlying the skin.
17. The apparatus according to inventive concept 16, wherein the first and second support structures are configured to be disposed generally parallel to one another in a manner in which the first support structure is disposed at one side of the portion of skin and underlying tissue, and the second support structure is disposed at an opposite side of the portion of skin and underlying tissue.
18. The apparatus according to inventive concept 17, wherein:

the first support structure is shaped so as to define a lateral surface at an interface between the first support structure and the portion of skin and underlying tissue disposed in part within the at least a part of the housing, and

the apparatus further comprises an acoustic lens shaped to provide a first lateral surface and a second lateral surface having two or more concave surfaces, and wherein the acoustic lens is configured to be disposed between the ultrasound transducer and a first surface of the portion of skin and underlying tissue in a manner in which:
the first lateral surface of the acoustic lens is in acoustic communication with the ultrasound transducer, and
the second lateral surface of the acoustic lens is in acoustic communication with the lateral surface of the first support structure and with the first surface of the portion of skin and underlying tissue.
   19. The apparatus according to inventive concept 17, wherein the first lateral surface of the lens is planar.
   20. The apparatus according to inventive concept 17, wherein the second lateral surface of the acoustic lens comprises a plurality of concave surfaces, and wherein each concave surface is configured to focus the energy transmitted from the ultrasound transducer toward a respective focal zone in the first surface of the portion of skin and underlying tissue.
   21. The apparatus according to inventive concept 17, wherein the at least one acoustic lens is moveable with respect to the first support structure along the axis that is not parallel to the lateral surface of the first support structure.
   22. The apparatus according to inventive concept 17, wherein each one of the two or more concave surfaces of the second lateral surface of the acoustic lens is configured to focus the energy toward a respective focal zone in the first surface of the portion of skin and underlying tissue.
   23. The apparatus according to inventive concept 22, wherein each one of the two or more concave surfaces of the second lateral surface of the acoustic lens is configured to facilitate generating of a respective hole at each focal zone in the first surface of the portion of skin and underlying tissue.
   24. The apparatus according to inventive concept 17, wherein:

the first support structure is shaped so as to define a lateral surface at an interface between the first support structure and the portion of skin and underlying tissue disposed in part within the at least a part of the housing,

the ultrasound transducer is moveable with respect to the first support structure along an axis of the first support structure that is not parallel to that the lateral surface of the first support structure, and

the ultrasound transducer is coupled to the first support structure in a manner in which it is in acoustic communication with the lateral surface of the first support structure.

25. The apparatus according to inventive concept 24, wherein:

the first support comprises a track disposed along the axis of the first structure, and

the ultrasound transducer is moveably coupled to the track.

26. The apparatus according to inventive concept 24, wherein:

the second support structure is shaped so as to define a lateral surface at an interface between the second support structure and the portion of skin and underlying tissue disposed in part within the at least a part of the housing,

the acoustic element is moveable with respect to the second support structure along an axis of the second support structure that is not parallel to the lateral surface of the second support structure, and

the acoustic element is coupled to the second support structure in a manner in which is in acoustic communication with the lateral surface of the second support structure.

27. The apparatus according to inventive concept 26, wherein:

the second support structure comprises a track disposed along the axis of the second support structure, and

the acoustic element is moveably coupled to the track.

28. The apparatus according to inventive concept 26, wherein:

the ultrasound transducer is moveable with respect to the first support structure along the axis that is not parallel to the lateral surface of the first support structure,

the ultrasound transducer comprises a first ultrasound transducer configured to transmit energy toward the portion of skin and underlying tissue in a manner in which a first focal zone is created in the portion of skin and underlying tissue, and

the first transducer is configured to alter a position of the first focal zone by being moved along the axis.

29. The apparatus according to inventive concept 28, wherein:

the acoustic element is moveable with respect to the second support structure along the axis that is not parallel to the lateral surface of the second support structure,

the acoustic element defines a second ultrasound transducer configured to transmit energy toward the portion of skin and underlying tissue such that a second focal zone is generated in the portion of skin and underlying tissue, and

the second transducer is configured to alter a position of the second focal zone by being moved along the axis.

30. The apparatus according to inventive concept 29, wherein the first and second ultrasound transducers are arranged such that transmission of energy from the first and second ultrasound transducers causes the first and second focal zones to overlap at least in part.
31. The apparatus according to inventive concept 29, wherein the first and second ultrasound transducers are arranged such that transmission of energy from the first and second ultrasound transducers causes the first and second focal zones to move within the portion of skin and underlying tissue in accordance with movement of the first and second ultrasound transducers.
32. The apparatus according to inventive concept 26, wherein:

the apparatus further comprises a first acoustic lens configured to be disposed between the ultrasound transducer and the portion of skin and the underlying tissue within the at least a part of the housing, and

the first lens is moveable with respect to the first support structure along the axis that is not parallel to the lateral surface of the first support structure.

33. The apparatus according to inventive concept 32, wherein the first lens is configured to alter a position of the first focal zone by being moved along the axis of the first support structure.
34. The apparatus according to inventive concept 32, wherein the first lens is configured to contact the skin and to be easily removable, by a user of the apparatus, from other components of the apparatus.
35. The apparatus according to inventive concept 32, wherein the acoustic lens is shaped to provide a first lateral surface and a second lateral surface having two or more concave surfaces, and wherein the acoustic lens is configured to be disposed between the ultrasound transducer and a first surface of the portion of skin and underlying tissue in a manner in which:

the first lateral surface of the acoustic lens is in acoustic communication with the ultrasound transducer, and

the second lateral surface of the acoustic lens is in acoustic communication with the lateral surface of the first support structure and with the first surface of the portion of skin and underlying tissue.

36. The apparatus according to inventive concept 35, wherein the first lateral surface of the acoustic lens is planar.
37. The apparatus according to inventive concept 35, wherein the second lateral surface of the acoustic lens comprises a plurality of concave surfaces, and wherein each concave surface is configured to focus the energy transmitted from the ultrasound transducer toward a respective focal zone in the first surface of the portion of skin and underlying tissue.
38. The apparatus according to inventive concept 35, wherein the at least one acoustic lens is moveable with respect to the first support structure along the axis that is not parallel to the lateral surface of the first support structure.
39. The apparatus according to inventive concept 35, wherein each one of the two or more concave surfaces of the second lateral surface of the acoustic lens is configured to focus the energy toward a respective focal zone in the first surface of the portion of skin and underlying tissue.
40. The apparatus according to inventive concept 39, wherein each one of the two or more concave surfaces of the second lateral surface of the acoustic lens is configured to facilitate generating of a respective hole at each focal zone in the first surface of the portion of skin and underlying tissue.
41. The apparatus according to inventive concept 32, wherein:

the apparatus further comprises a second acoustic lens configured to be disposed between the second acoustic element and the portion of skin and the underlying tissue within the at least a part of the housing,

the second lens is moveable with respect to the second support structure along the axis of the second support structure, and

the second lens is configured to focus the transmitted energy to at least one second focal zone.

42. The apparatus according to inventive concept 41, wherein the second lens is configured to alter a position of the second focal zone by being moved along the axis of the second support structure.
43. The apparatus according to inventive concept 41, wherein the first and second lenses are configured to focus the transmitted energy toward the portion of skin and underlying tissue in a manner in which the first and second focal zones overlap at least in part.
44. The apparatus according to inventive concept 41, wherein the first and second lenses are arranged in manner in which the second focal zone generated in the portion of skin and the underlying tissue substantially midway between the ultrasound transducer and the acoustic element.
45. The apparatus according to inventive concept 41, wherein the second lens is configured to contact the skin and to be easily removable, by a user of the apparatus, from other components of the apparatus.
46. The apparatus according to inventive concept 1, wherein the ultrasound transducer and the acoustic element are configured to operate in generally closed looped operation.
47. The apparatus according to inventive concept 46, wherein the processing unit is configured to monitor the alteration of the parameter and regulate the transmission of the treatment energy in response to the monitoring.
48. The apparatus according to inventive concept 46, wherein the ultrasound transducer and the processing unit are configured to cycle repeatedly between (a) applying a treatment to the tissue in response to the monitored state of the tissue, and (b) monitoring the state of the tissue following (a).
49. A method, comprising:

placing on skin of a subject:
at least one ultrasound transducer, the ultrasound transducer being configured to transmit through the skin one or more forms of acoustic radiation, at least one of the one or more forms of energy including treatment energy, and
at least one acoustic element with respect to the ultrasound transducer such that the one or more forms of energy are through-transmitted toward the acoustic element;

drawing at least a portion of the skin and underlying tissue between the at least one ultrasound transducer and the at least one acoustic element;

transmitting from the at least one ultrasound transducer one or more forms of energy through the portion of skin and the underlying tissue;

generating confocal transmission responsively to the transmitting;

receiving at least a portion of the through-transmitted energy at least one component selected from the group consisting of: the at least one ultrasound transducer and the at least one acoustic element;

monitoring a change in a parameter of the tissue underlying the skin responsively to the received energy; and

responsively to the monitoring, applying a treatment to the apply a treatment to the portion of skin and underlying tissue.

50. The method according to inventive concept 49, wherein transmitting the energy comprises generating at least one fixed focal zone in the portion of skin and underlying tissue.
51. The method according to inventive concept 49, wherein transmitting the energy comprises generating at least one moveable focal zone in the portion of skin and underlying tissue.
52. The method according to inventive concept 49, wherein transmitting the energy comprises generating a standing wave in the portion of skin and underlying tissue.
53. The method according to inventive concept 49, wherein transmitting the energy comprises generating constructive interference in the portion of skin and underlying tissue.
54. The method according to inventive concept 49, wherein transmitting the energy comprises generating an imploding wave in the portion of skin and underlying tissue.
55. The method according to inventive concept 49, wherein the ultrasound transducer is moveably coupled to a first support structure, and wherein the method further comprises moving the at least one ultrasound transducer with respect to the first support structure along an axis that is perpendicular to a lateral surface of the first support structure.
56. The method according to inventive concept 55, wherein moving the at least one ultrasound transducer with respect to the first support structure comprises moving the at least one ultrasound transducer along a track coupled to the first support structure.
57. The method according to inventive concept 55, wherein moving the at least one ultrasound transducer comprises:

moving the ultrasound transducer while keeping the first support structure in place, and

moving a focal zone generated by the ultrasound transducer.

58. The method according to inventive concept 49, wherein the acoustic element is moveably coupled to a second support structure, and wherein the method further comprises moving the at least one acoustic element with respect to the second support structure along an axis that is perpendicular to a lateral surface of the second support structure.
59. The method according to inventive concept 58, wherein moving the at least one acoustic element with respect to the second support structure comprises moving the at least one acoustic element along a track coupled to the second support structure.

The present invention will be more fully understood from the following detailed description of applications thereof, taken together with the drawings, in which:

### BRIEF DESCRIPTION OF THE DRAWINGS

FIG. 1 is a schematic illustration of apparatus comprising an ultrasound device, positioned on tissue of a subject, in accordance with some applications of the present invention;

FIG. 2 is a schematic illustration of apparatus comprising an ultrasound device, positioned on tissue of the subject, in accordance with some other applications of the present invention;

FIGS. 3A-B are schematic illustrations of apparatus comprising an ultrasound device comprising acoustic lenses, in accordance with respective applications of the present invention;

FIG. 4 is a schematic illustration of apparatus comprising an ultrasound device, in accordance with yet another application of the present invention; and

FIG. 5 is a schematic illustration of apparatus comprising an ultrasound device, in accordance with still another application of the present invention.

### DETAILED DESCRIPTION OF THE APPLICATIONS

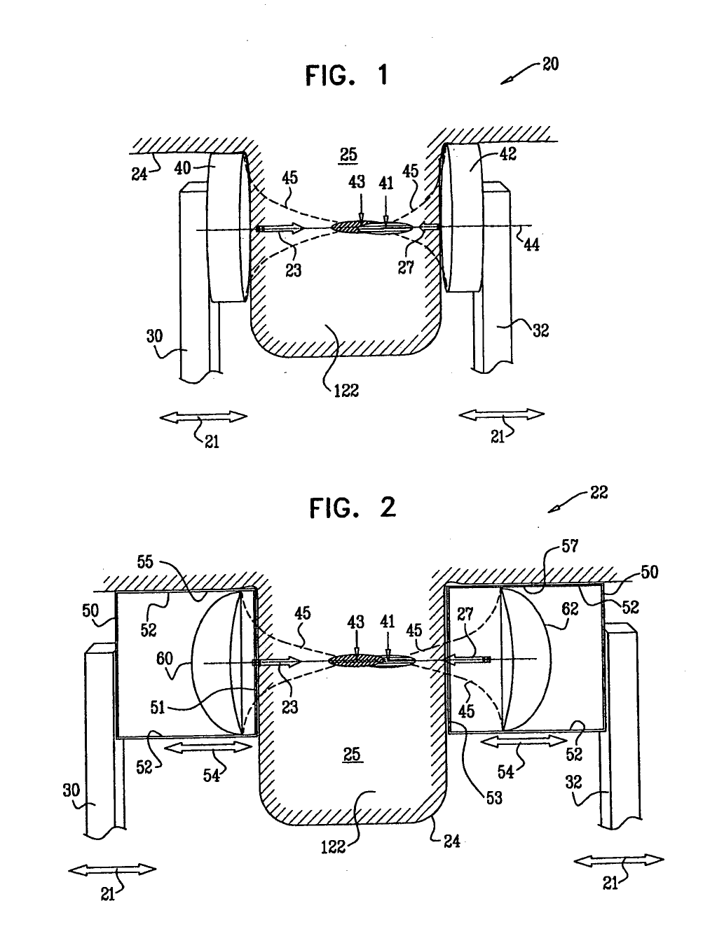

Reference is now made to FIG. 1, which is a schematic illustration of apparatus 20 comprising a first acoustic element 40 and a second acoustic element 42, in accordance with some applications of the present invention. Typically, first and second acoustic elements 40 and 42 comprise respective first and second phased arrays of ultrasound transducers. For some applications of the present invention, first and second acoustic elements 40 and 42 each comprise at least a single ultrasound transducer. For some applications of the present invention, the transducer comprises a non-focused flat transducer. For some applications of the present invention, the transducer comprises a transducer having a curved surface which faces and is in acoustic communication with the portion of tissue and focuses the energy toward the portion of tissue. Apparatus 20 comprises a housing, e.g., a c-shaped clamp, comprising a horizontal support element (not shown) coupled to a first support structure 30 and a second support structure 32. As shown, support structures 30 and 32 are rectangular, by way of illustration and not limitation. First and second support structures 30 and 32 are disposed not parallel, e.g., perpendicularly, to the horizontal support element and are spaced apart from each other at a distance ranging from about 5 mm to about 150 mm, e.g., about 5 mm to 40 mm or 40 mm to 150 mm. At least one, e.g., both, of support structures 30 and 32 is configured to move axially with respect to the horizontal support element in a direction as indicated by arrows 21. Typically, support structures 30 and 32 comprise first and second pinching elements, respectively, configured to pinch a portion of the skin therebetween to draw the portion into a plane defined by the housing.

For some applications of the present invention, support structures 30 and 32 comprise cylinders. One or both of support structures 30 or 32 move axially or rotate in such a manner so as to pinch and draw a portion 122 of tissue, or area designated for treatment and/or monitoring, between supports 30 and 32. Alternatively or additionally, the cylinders rotate in the same direction or in opposite directions, to draw new tissue into the plane.

In either application, the movement and distances between acoustic elements 40 and 42 or portions thereof are typically recorded by a linear encoder or by counting steps of a stepper motor. Such recording is useful in the monitoring of a body contouring process, as described hereinbelow. Additionally, such recording is useful in facilitating calculating speed of sound (SOS), as described hereinbelow.

First acoustic element 40 is coupled to first support structure 30, and second acoustic element 42 is coupled to second support structure 32. Support structures 30 and 32 maintain acoustic elements 40 and 42, respectively, in a desired relationship with respect to each other. Prior to application of treatment energy, the housing is placed on skin 24 of the subject such that (a) support structures 30 and 32 are aligned substantially perpendicularly with respect to a surface of skin 24, and (b) at least respective portions of acoustic elements 40 and 42 contact skin 24. At least one, e.g., both, of the support structures 30 and 32 is moveable with respect to the second so as to draw, e.g., pinch or clamp, a tissue portion 122 including skin 24 and underlying tissue 25, within an area between support structures 30 and 32. Such an area defines a plane 44 of the housing. Alternatively or additionally, the housing comprises a source of suction (not shown), to draw portion 122 of skin 24 and underlying tissue 25 between structures 30 and 32, e.g., within at least a portion of the housing, in a manner as shown in FIG. 1. In such an application, the source of suction would be coupled to the housing in a space defined by the housing that is between the first and second support structures. Typically, following the drawing of the tissue between support structures 30 and 32, good acoustic coupling is verified between the skin 24 and tissue 25 within tissue portion 122 and acoustic elements 40 and 42, prior to acoustic elements 40 and 42 entering a monitoring mode. Such verification may be performed, for example, by transmitting “scout” waves from one support structure to the other. Following the drawing of tissue portion 122 between support structures 30 and 32, ultrasound waves are transmitted from acoustic elements 40 and 42 in a treatment mode toward the center of tissue portion 122, e.g., equidistant from (a) an interface between acoustic element 40 and skin 24 of tissue portion 122, and (b) an interface between acoustic element 42 and skin 24 of tissue portion 122. For some applications of the present invention, waves are transmitted from acoustic elements 40 and/or 42 in a manner in which at least one focal zone is generated in the portion of skin and the underlying tissue substantially midway between acoustic elements 40 and 42. It is to be noted that substantially midway between elements 40 and 42 includes the center of portion 122 as well as a majority of portion 122 which excludes the hatched portions which represents the stratum corneum.

For some applications of the present invention, each support structure 30 and 32 is surrounded at least in part by a disposable membrane. For example, a membrane is coupled to the respective portions of each support structure 30 and 32 at their interface with the skin of the subject. The membrane may then be easily removable, by a user of the apparatus, from other components of the apparatus.

During the treatment mode, some or all of the ultrasound transducers of the respective phased arrays of acoustic elements 40 and 42 transmit high intensity focused waves simultaneously or in a temporal pattern, toward the center of tissue portion 122. Typically, the energy is transmitted at a frequency of between 2 MHz and 20 MHz, e.g., between 3 MHz and 5 MHz. Energy is transmitted toward the center of portion 122 in a manner in which (a) the intensity at the surface of skin 24 does not exceed 3 Watts/cm̂2, and (b) the intensity at focal points 41 and 43 is, by way of illustration and not limitation, between 10 Watts/cm̂2 and 300 Watts/cm̂2, e.g., between 10 Watts/cm̂2 and 100 Watts/cm̂2, typically 15 Watts/cm̂2. This typically creates high intensity, focused ultrasound waves such as imploding waves or imploding cylindrical waves, whose amplitude (positive or negative) is high at the center. Consequently, damage to the tissue occurs relatively rapidly in and around focal points 41 and 43. For some applications of the present invention, the imploding wave is generated while substantially avoiding cavitation within the treatment area of tissue portion 122. The implosion waves are then inwardly directed toward the center of tissue portion 122.

Typically, parameters of treatment energy to only warm tissue comprises applying, by way of illustration and not limitation, between 0.5 Watts/cm̂2 and 3 Watts/cm̂2, e.g., 3 W/cm̂2.

Alternatively, other signal protocols create other ultrasound-based effects besides an imploding cylindrical wave, which, nevertheless, produce a desired level of tissue damage. In any case, following the transmission of the energy from acoustic element 40 and/or acoustic element 42, the apparatus is typically switched back to the monitoring mode and damage assessment is performed. If appropriate, another iteration of high energy transmission is performed, followed by another iteration of monitoring (entirely automated, or human supported). The procedure is repeated until satisfactory results are obtained. At this point, tissue portion 122 is released from being clamped between support structures 30 and 32 and the operator or the robotic system moves the device to a new region to be treated, optionally based on feedback from the monitoring.

For some applications of the present invention, support structures 30 and/or 32 are manually movable by the ultrasound technician and can be used independently of the housing and/or of the horizontal support structure. Alternatively or additionally, the housing comprises an electromechanical system which moves support structures 30 and/or 32 with respect to each other. The electromechanical system is typically coupled to the housing and for some applications generates suction within the housing. Optionally, the electromechanical system dispenses ultrasound gel to enhance acoustic coupling with the tissue. Alternatively or additionally, the electromechanical system dispenses water for cooling the device or tissue. Further alternatively or additionally, a portion of the housing comprises a reservoir (not shown) of water and/or gel, for dispensing by an operator during a procedure.

Once portion 122 of the tissue is drawn between acoustic elements 40 and 42, portion 122 of the tissue is monitored to assess a parameter of the tissue, e.g., fat content, by transmitting low intensity ultrasound waves from acoustic element 40 and/or 42. Following the initial monitoring of the skin, acoustic elements 40 and/or 42 transmit treatment energy toward the center of portion 122. Monitoring of the tissue is typically performed in conjunction with the applied treatment energy. For some applications of the present invention, acoustic elements 40 and 42 operate in a closed loop operation, cycling between treatment and monitoring. For some applications of the present invention, acoustic elements both apply treatment energy and monitor the parameter of tissue 25 and skin 24 in tissue portion 122.

Typically, acoustic element 40 comprises at least one ultrasound transducer, e.g., a phased array of ultrasound transducers, configured to transmit energy in a plurality of modes (e.g., treatment or monitoring) in accordance with a plurality of ultrasound protocols. Acoustic element 40 transmits energy toward acoustic element 42 which either receives and/or reflects through-transmitted and/or scattered energy back in the direction of acoustic element 40. For applications in which acoustic element 42 receives the through-transmitted and/or scattered energy, acoustic element 42 comprises at least a single ultrasound transducer which receives the energy and transmits the received energy to a processing unit, which detects, digitizes, and analyzes the received energy and monitors portion 122 of the tissue responsively to the receiving. For applications in which acoustic element 42 comprises a phased array of ultrasound transducers, at least a portion of the transducers in the array receive the through-transmitted energy from acoustic element 40. For some applications, acoustic element 42 also transmits energy toward acoustic element 40, which either receives and/or reflects the through-transmitted energy back in the direction of acoustic element 42. In either application, elements 40 and 42 receive scattered energy from portion 122. In some applications, elements 40 and/or 42 comprise acoustic reflectors.

For some applications of the present invention, acoustic element 40 comprises at least a single ultrasound transducer configured to transmit treatment energy in plane 44 toward the center of portion 122 of tissue 25. For some applications of the present invention, the ultrasound energy is reflected from acoustic element 42 back toward acoustic element 40. In such an application, acoustic element 40 receives the through-transmitted and/or scattered ultrasound energy and transmits the received energy to a processing unit which digitizes and analyzes the received energy and monitors portion 122 of the tissue responsively to the receiving.

As shown in FIG. 1, acoustic element 40 transmits energy in a direction as indicated by arrow 23, and acoustic element 42 transmits energy in a direction as indicated by arrow 27. For some applications of the present invention, acoustic elements 40 and 42 each comprise an annular phased array of concentrically-disposed ring-shaped ultrasound transducers which each transmit acoustic radiation beams 45 (e.g., in a pulse/burst mode or a continuous wave mode) toward the center of portion 122 of tissue 25. The phased array of acoustic element 40 transmits beams 45 toward a first focal zone 43, and the phased array of acoustic element 42 transmits beams 45 toward a second focal zone 41. Typically, focal zones 41 and 43 overlap at least in part to create confocal transmission in the center of portion 122 of tissue 25. Each acoustic element of the respective annular phased arrays of ultrasound transducers of elements 40 and 42 is activated independently with a specific phase and amplitude. Using known techniques for beam-forming, acoustic beam 45 transmitted from each transducer in the array can be axially steered and focused at the center of the clamped tissue portion 122.

In such an application, the target treatment site receives energy confocally from both acoustic elements 40 and 42, thus achieving the optimum intensity at the center of portion 122 in a shorter period of time (e.g., half the time) than if only one acoustic element transmitted energy toward the center of tissue 25 (while minimizing heating of non-targeted tissues) in a system comprising only the one ultrasound transducer. Because the treatment site is receiving confocal acoustic beams from either direction, the intensity of energy transmitted through skin 24 is distributed between the portion of the skin adjacent to acoustic element 40 and the portion of the skin adjacent to acoustic element 42. In such an application, the intensity at the surface of the skin is reduced by half (with respect to the intensity at the surface of the skin in techniques where only one acoustic element is used) when using confocal acoustic radiation techniques, thereby minimizing damage at the surface of skin 24.

Typically, the following effects occur when applying confocal radiation to tissue portion 122: (a) the radiation intensity at the mutual focal zone (i.e., the overlapping portions of focal zones 41 and 43) can be doubled, thereby reducing the treatment time, (b) the intensity at the surface of skin 24 is reduced because the transmitted energy is split over two surface of the portion of skin 24 disposed adjacently to acoustic elements 40 and 42, respectively, (c) a first acoustic element can be used as a treatment element, and a second acoustic element can be used as a monitoring element for monitoring treatment responsively to energy transmitted from the first acoustic element, (d) reflected waves are re-focused at the focal zones 41 and 43, and (e) the confocal system sets the focal zones at the center clamped/pinched tissue portion 122, thus avoiding potential skin injury.

Monitoring of the tissue in conjunction with the application of the treatment energy is accomplished when at least one ultrasound transducer transmits energy toward a second ultrasound transducer which receives a portion of the transmitted energy and passively detects echoes from the first transducer in combination with techniques for passive beam-forming (e.g., in order to modulate the spatial sensitivity of the detecting transducer). Confocality is achieved in these applications when the intensity of the through-transmitted energy detected by either of acoustic elements 40 and 42 is maximal. Typically, confocality is achieved by first transmitting acoustic beams 45 from acoustic element 40. Through-transmitted energy is detected by acoustic element 42. Acoustic element 42 then transmits energy to be detected by acoustic element 40. Once overlapping focal zones are achieved and detected, i.e. the overlap between the two focal zones is maximal, the two elements 40 and 42 transmit high intensity treatment energy simultaneously or alternately. During the treatment, one of acoustic elements 40 or 42 can be switched into receiving mode and the detected signal can be used for determining the progress of the treatment (e.g., by analyzing acoustic properties of the tissue and generating maps of the acoustic properties of the tissue, or by evaluating the temperature at the focal zone).

Beams 45 transmitted from the respective annular phased arrays of acoustic elements 40 and 42 are capable of being electronically steered so as to change the focal zone within portion 122 of tissue 25. The steering is typically performed without physically moving support structures 30 and 32. The steering of the focal zone facilitates concentration of the energy generally at the center of portion 122. Typically, the through-transmitted energy from acoustic element 40 is, in part, reflected by acoustic element 42, and vice versa. The reflected energy is then refocused toward the center of portion 122 of tissue 25 so as to supplement the confocal acoustic radiation provided by acoustic elements 40 and 42. In such an application, the arrays are capable of varying their respective focal points in response to an electronic system which electronically reconfigures the transducers in the arrays. Additionally, each of the first and second arrays reflects through-transmitted energy back toward the respective opposing array and toward the center of the portion of the skin and underlying tissue that is drawn between the first and second support structures.

Additionally, using the confocal techniques described herein, acoustic element 40 transmits treatment energy while acoustic element monitors the parameter of portion 122 of tissue 25, and vice versa.

Acoustic elements 40 and 42 are typically connected via coupling lines to a workstation (not shown) which is configured to drive and receive data from acoustic elements 40 and 42. The workstation processes signals from acoustic elements 40 and 42 in order to generate acoustic maps or images of tissue portion 122. The resultant maps or images indicate whether a desired extent of treatment has been obtained (e.g., a level of damage to tissue), and guide further treatment.

It is noted that although some applications of the present invention are described herein with respect to generally closed-loop operation of acoustic elements 40 and 42, the scope of the present invention includes the use of acoustic elements 40 and 42 only for monitoring the tissue, while, for example, another device (e.g., a prior art ultrasound device) applies a treatment. Similarly, the scope of the present invention includes the use of acoustic elements 40 and 42 only for treating the tissue, while, for example, another device (e.g., a prior art ultrasound device) monitors the progress of the treatment. Alternatively, only monitoring is performed, or only treatment is performed.

For some applications of the present invention, a robotic system moves the housing to different sites on skin 24.

Maps of acoustic properties or images of the tissue area are reconstructed, typically using algorithms that are known in the art. As appropriate, the maps or images may depict various acoustic properties of the tissue, such as reflectivity, time of flight, speed of sound, attenuation, acoustic impedance, and other properties. For some applications, the maps or images thus acquired are saved for later use as a reference set. For some applications of the present invention, maps of acoustic properties are translated into maps that show tissue type within tissue portion 122, and, for example, differentiate between fat tissue and muscle, nerve or blood cell tissues. Alternatively or additionally, maps of acoustic properties are translated into temperature maps, e.g., using techniques described in the above-cited PCT Publication WO 06/018837 to Azhari et al., which is incorporated herein by reference, and/or using other techniques known in the art. Further alternatively or additionally, maps of acoustic properties are assessed by computer or by a human to determine the efficacy of the treatment, and are saved or used to modify further treatments.

For some applications of the present invention, an external source of energy is used to treat tissue portion 122 drawn, or clamped, between support structures 30 and 32. For some applications in which the external energy source is applied, acoustic elements 40 and 42 typically work only in the monitoring mode. Maps or images are typically acquired generally continuously during the treatment. The changes derived from the treatment result in changes of the detected acoustic properties of the treated tissue. By subtracting the new maps or images from the reference set of maps or images, the amount and location of damage is assessed. Alternatively, the reference set is not used, but instead a desired endpoint is designated, and a signal is generated when the endpoint is approached or attained.

For applications in which acoustic elements 40 and 42 are used in combination with the external energy source, the external energy source may be coupled to the housing, or, alternatively, mechanically separate from the housing (configuration not shown). The external energy source comprises circuitry for focusing energy designated for the destruction of adipose tissue, such as acoustic energy (e.g., high intensity focused ultrasound, shock waves, sharp negative pressure pulses, or high intensity ultrasound waves), electromagnetic radiation (e.g., microwave radiation or radiofrequency), laser energy, and/or visual or near-visual energy (e.g., infra-red). The external energy source transmits energy intense enough to cause damage to adipose tissue within portion 122.

Typically, effects of treatment energy applied by acoustic elements 40 and/or 42 independently of or in combination with effects of treatments by the external energy source may include, as appropriate, heating, tissue damage, thermal ablation, mechanical irritation, acoustic streaming, cell structure alteration, augmented diffusion, and/or a cavitation effect. For some applications, lipolysis is accomplished when the temperature of portion 122 of tissue 25 is elevated by less than 10 C, e.g., less than 5 C.

For some applications, acoustic elements 40 and/or 42 independently of or in combination with effects of treatments by the external energy source, provide energy such that the treatment generates a combined effect of at least two of the above-mentioned effects. For this application, energy is applied, inducing a different type of damage to the tissue. The sets are typically operated in a synchronized mode to enhance the tissue damaging process. Alternatively, a multipurpose array is used which is capable of producing at least two types of damage to a predefined tissue region by applying a plurality of transmissions (e.g., a sequence of transmissions or parallel transmissions). Inducing the at least two types of damage simultaneously or alternately creates synergism, accelerating the tissue damaging procedure and reducing the overall treatment time.

In accordance with some applications of the present invention, acoustic elements 40 and/or 42 are switched to a treatment mode, typically a plurality of times in alternation with the monitoring mode described hereinabove. In the treatment mode, acoustic elements 40 and/or 42 transmit high intensity ultrasound waves, shock waves, sharp negative pressure pulses, continuous waves (CW), pulse sequences that cause cavitation, any other form of acoustic radiation that affects the tissue in a desired manner, or any combination of the above. Typically, but not necessarily, the ultrasound transducers transmit the energy in a HIFU mode.

It is noted that by using confocal transmission by phased array techniques, the phase of the transmitted waves from each ultrasound acoustic element 40 and 42 can be controlled such that the focal point of the imploding wave is moved over a significant portion of the area within tissue portion 122, without physically moving support structures 30 and 32. The timing of transmission of the ultrasound wave from each acoustic element 40 and/or 42 is set such that wave fronts transmitted from element 40 and/or 42 arrive at the focal point with generally the same phase, creating a sharp local peak in intensity which causes thermal and mechanical damage to tissue 25 in portion 122.

For some applications, in addition to monitoring the treatment procedure, the body contouring process is tracked by sensors (not shown) that are coupled to the housing. For example, the sensors may comprise electromagnetic sensors or optical sensors. The sensed information is transmitted to the processing unit. Storing the tracking information allows for improved follow-up and comparison of body contouring treatments conducted on different days or during the treatment.

For some applications, tracking the treatment process occurs in conjunction therewith. In response to an indication of fat content detected by the acoustic elements in a particular area of the body of the subject, a pre-treatment map is generated and the physician marks the area, designating it for treatment. The housing is subsequently placed on the designated area to provide treatment and monitoring thereof. Following the treatment, the housing is re-positioned in the designated area to enable tracking of the body contouring process by the sensors. The sensors help ensure that (1) treatment has been applied to all subsections of the designated area and/or (2) treatment has not been applied multiple times to the same subsection during a single session. Thus, for some applications, treatment locations during one session are stored to facilitate the initiation of treatments in subsequent locations other than already-treated regions.

Typically, monitoring by the acoustic elements is accomplished by a series of low intensity ultrasonic pulses transmitted from a portion of acoustic elements of subset 30. It is to be noted that other waveforms can be utilized. The energy is scattered by, reflected by, or transmitted through portion 122 of tissue 25. At least a portion of the energy is then received by acoustic element 40 and/or 42, which is designated for monitoring the procedure. This portion of the energy is received by acoustic element 40 and/or 42, and travel times of pulses between acoustic elements 40 and 42 (T₁) are calculated, using techniques known in the art. The amplitudes (Amp₁) of echoes received by acoustic elements 40 and 42 are also registered.

The average speed of sound (SOS) is calculated as follows:

SOS=L/T₁, where L represents a distance between acoustic elements 40 and 42. Distance L is recorded by a linear encoder, a stepper motor or another device known in the art and configured to detect and/or sense and digitize linear position change for position measurement and feedback to the monitoring system, in order to calculate and monitor the SOS.

The average attenuation coefficient (mu) is calculated as follows:

mu=Log(Amp₁/Amp₀),

where Amp₀ is a reference amplitude.

In addition, the spectrum of both reflected waves (S_{R}) and the spectrum of the transmitted waves (S_{T}) are analyzed.

Using the properties SOS, mu, S_{R} and S_{T}, portion 122 of tissue 25 is characterized to assess whether the concentration of fat in portion 122 is sufficient for application of treatment energy thereto. Once treatment energy has been applied to portion 122, changes in the properties of SOS, mu, S_{R} and S_{T} are monitored. Expected changes as a result of the treatment process (e.g., elevated temperature, appearance of cavitation bubbles, and changes in the cellular structure) are manifested in and alter the acoustic properties SOS, mu, S_{R} and S_{T}. For example, it is known that SOS and mu change with temperature, and that the appearance of cavitation bubbles induces half harmonic signals in the spectrum relating to the reflected and transmitted waves. Methods described herein may be practiced in combination with methods for assessing a parameter of tissue described in the above-mentioned articles by Moran et al. and Akashi et al., which are incorporated herein by reference.

In some applications, when a distance between elements 40 and 42 is kept constant (i.e., elements 40 and 42 do not slide along the axes of the first and second support structures during the time of monitoring), the time of flight (TOF) of the acoustic radiation energy is measured and can be used equivalently to SOS for monitoring the treatment.

The acoustic elements 40 and 42 comprise at least one ultrasound transducer which receives the scattered and through-transmitted echoes and transmits the received energy to the processing unit of the workstation. The workstation is configured to drive and receive data from the transducers. The workstation processes signals from the transducers in order to generate acoustic maps or images (e.g., a local B-scan image generated by the echoes or an image generated by through-transmission) of portion 122 of tissue 25 that is enclosed in the plane. The resulting maps or images indicate whether a desired extent of treatment has been obtained (e.g., a level of damage to tissue 25) and guide further treatment. Cycles of treatment and monitoring occur in a generally closed-loop manner and are repeated using different signaling parameters, until a sufficient amount of data is collected. Maps of acoustic properties or images of the tissue are reconstructed and assessed.

It is to be noted that since tissue 25 to be treated includes adipose tissue, for some applications the registered information is calibrated to provide tables relating the intensity of treatment to the expected changes in each of the acoustic properties: SOS, mu, S_{R} and S_{T}. When the desired effect has been achieved, the treatment is terminated.

For some applications, during the monitoring of the concentration of fat in tissue portion 122, acoustic elements 40 and 42 are moved such that two images of tissue portion 122 are obtained. The first image is reconstructed from the reflected echoes depicting a standard B-scan image, and the other image is reconstructed from the through-transmitted waves, using ultrasonic tomography algorithms known in the art, e.g., Back-Projection-based methods. The second image may depict a map of SOS and/or mu in the imaged region.

Reference is now made to FIG. 2, which is a schematic illustration of apparatus 22 comprising a first acoustic element 60 comprising a focused ultrasound transducer shaped to define a curved surface in communication with a lateral surface 51 of a surrounding case 50 of structure 30, and a second acoustic element 62 comprising a focused ultrasound transducer shaped to define a curved surface in communication with a lateral surface 53 of case 50 of structure 32, in accordance with some applications of the present invention. Acoustic elements 60 and 62 are disposed within respective cases 50 that are each coupled to and comprise a portion of support structures 30 and 32, respectively. Each case 50 defines respective portions of structures 30 and 32 and has a respective lower surface 55 and 57 and a respective lateral surface 51 and 53. Lateral surfaces 51 and 53 of each case 50 are configured to be disposed at respective interfaces between first and second support structures 30 and 32 with portion 122 of skin and underlying tissue. Lower surfaces 55 and 57 are configured to contact the surface of the skin of the subject at portions thereof that are not disposed between structures 30 and 32.

Typically, acoustic elements 60 and 62 each comprising focused transducers which each comprise transducers having a concave surface in communication with lateral surfaces 51 and 53 of support structures 30 and 32, respectively. Each focused transducer transmits ultrasound beams 45, e.g., in a pulse/burst mode or a continuous wave mode, toward respective focal zones 43 and 41 in order to create confocal transmission as described hereinabove with respect to FIG. 1. As described hereinabove, focal zones 41 and 43 overlap at least in part in the center of portion 122 of tissue 25 e.g., equidistant from (a) an interface between acoustic element 60 and skin 24 of tissue portion 122, and (b) from an interface between acoustic element 62 and skin 24 of tissue portion 122. For some applications of the present invention, waves are transmitted from acoustic elements 40 and 42 in a manner in which at least one focal zone is generated in the portion of skin and the underlying tissue substantially midway between elements 60 and 62. It is to be noted that substantially midway between elements 40 and 42 includes the center of portion 122 and a majority of portion 122 which excludes the hatched portions which represents the stratum corneum.

Typically, the focal zones of the focused ultrasound transducers defining acoustic elements 60 and 62 are fixed. Thus, in order to move respective focal zones 43 and 41 with respect to tissue portion 122, each element 60 and 62 slides axially. (i.e., in the directions as indicated by arrows 54) along a respective track 52 disposed within each case 50. Moving of focal zones 41 and 43 is in accordance with movement of either one or both acoustic element 60 and/or 62. Tracks 52 are disposed along respective axes of structures 30 and 32 that are not parallel, e.g., perpendicular, to lateral surfaces 51 and 53 of cases 50, and enable elements 60 and 62 to slide along the respective axes. Thus, elements 60 and 62 are moveable with respect to tissue portion 122 and with respect to support structures 30 and 32, respectively (i.e., while structures 30 and 32 remain in place with respect to the skin of the subject). Typically, cases 50 comprise an acoustic coupling medium (e.g., water or oil) which maintains acoustic coupling between tissue portion 122 and elements 60 and 62 as they are axially moved along tracks 52 within cases 50.

It is to be noted that tracks 52 are shown by way of illustration and not limitation, and that the desired movement of elements 60 and 62 may be accomplished without tracks, using any other suitable means known in the art.

For some applications of the present invention, the focused transducer defining element 62 is replaced by a curved acoustic reflector which reflects and focuses the through-transmitted acoustic radiation in a pulse-echo mode from the transducer defining element 60 back toward the center of tissue portion 122 and even back toward the transducer defining element 60. Typically, when the focal point of the acoustic reflector aligns with the focal zone of the transducer defining element 60, a maximal echo is generated.

Typically, acoustic elements 60 and 62 function in both treatment and monitoring modes as described hereinabove with respect to acoustic elements 40 and 42, with reference to respective applications of FIG. 1. It is to be noted that for some applications, at least one of acoustic elements 60 and/or 62 functions to reflect through-transmitted energy, or is replaceable with an acoustic reflector.

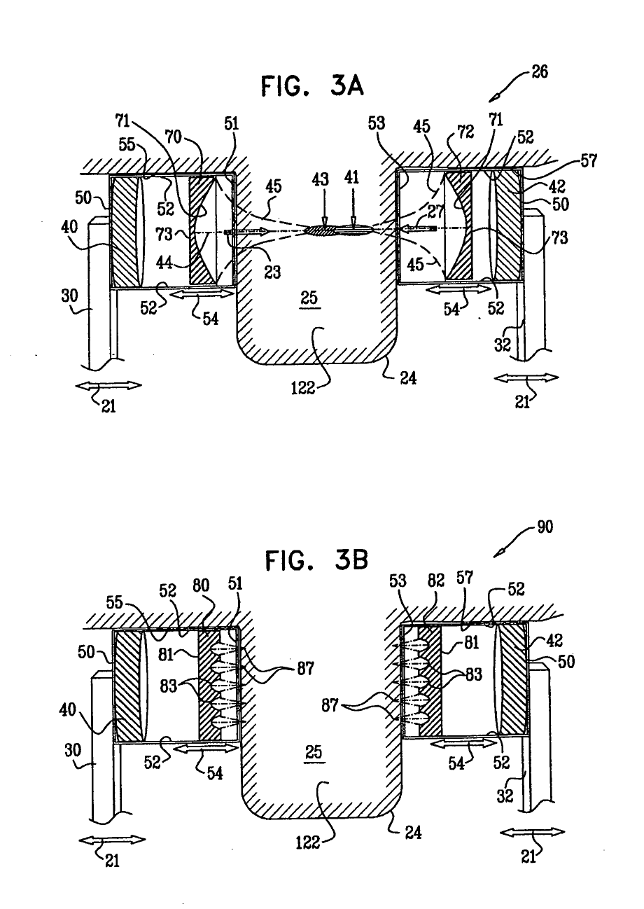

Reference is now made to FIG. 3A, which is a schematic illustration of apparatus 26 comprising a first acoustic element 40 comprising at least one ultrasound transducer and a second acoustic element 42 comprising at least one ultrasound transducer, in accordance with some applications of the present invention. The ultrasound transducers defining acoustic element 40 and 42 are disposed within respective cases 50 that are each coupled to support structures 30 and 32, respectively. For some applications, the transducers defining elements 40 and 42 each comprise a single non-focused transducer which transmits ultrasound beams 45 or pulses toward the center of tissue portion 122. Alternatively, elements 40 and/or 42 comprise focused transducers. For some applications of the present invention, the transducers defining elements 40 and 42 each comprise a respective phased array of ultrasound transducers.

A first acoustic lens, e.g., a plano-concave lens, 70 is disposed in acoustic communication between the transducer defining element 40 and tissue portion 122. A second acoustic lens, e.g., a plano-concave lens, 72 is disposed in acoustic communication between the transducer defining element 42 and tissue portion 122. Lenses 70 and 72 comprise a plastic, e.g., acrylic such as Perspex (Poly(methyl methacrylate) (PMMA)), by way of illustration and not limitation, and are each shaped to provide a planar surface, or interface 73, disposed in acoustic communication with the transducers defining elements 40 and 42, respectively, and a concave surface, or interface 71, that is inwardly curving in the direction of the transducers defining elements 40 and 42, respectively. Concave interfaces 71 face portion 122. For some applications of the present invention, lenses 70 and 72 are disposable and easily removable, by a user of the apparatus, from other components of the apparatus. Following each treatment, the lens may be replaced by another lens. In such an application, hygiene is maintained during application of the device to various subjects. For some applications of the present invention, interface 71 is coupled to or comprises polyurethane, by way of illustration and not limitation. Acoustic beams 45 transmitted from the transducers defining elements 40 and 42 are refracted by interfaces 71 and 73 of the lenses and are converged thereby toward focal zones 43 and 41, respectively. Apparatus 26 thus transmits acoustic energy toward respective focal zones 43 and 41 in order to create confocal transmission as described hereinabove with respect to FIGS. 1 and 2. As described hereinabove, focal zones 41 and 43 overlap at least in part in the center of portion 122 of tissue 25.

For some applications of the present invention, lenses 70 and 72 have identical curvatures of surfaces 71. For some applications of the present invention, lenses 70 and 72 have non-identical curvatures of surfaces 71. For some applications, lenses 70 and/or 72 are replaceable with lenses having a different curvature.

For some applications of the present invention, lenses 70 and 72 comprise lenses of various suitable geometries, as appropriate for a given application (e.g., ellipsoidal, spherical, zone lenses, etc.).

In either application, the focal zones of the non-focused transducers defining elements 40 and 42 are alterable. In order to move focal zones 41 and 43 with respect to tissue portion 122, each lens 70 and 72 slides axially (i.e., in the directions as indicated by arrows 54) along respective tracks 52 disposed within each case 50. Tracks 52 are disposed along respective axes of structures 30 and 32 that are not parallel, e.g., perpendicular, to lateral surfaces 51 and 53 of cases 50, and enable lenses 70 and 72 to slide along tracks 52 (e.g., while structures 30 and 32 remain in place with respect to the skin of the subject) and along the respective axes that are not-parallel to lateral surfaces 51 and 53 of cases 50. Thus, lenses 70 and 72 are moveable with respect to tissue portion 122 and with respect to lateral surfaces 51 and 53 of cases 50 of support structures 30 and 32, respectively. Typically, cases 50 comprise an acoustic coupling medium (e.g., water or oil) which maintains acoustic coupling between tissue portion 122, the transducers defining elements 40 and 42, and lenses 70 and 72 as they are axially moved along tracks 52 within cases 50.

It is to be noted that tracks 52 are shown by way of illustration and not limitation, and that the desired movement of lenses 70 and 72 may be accomplished without tracks, using any other suitable means known in the art. It is to be further noted that the transducers defining elements 40 and/or 42 may be moveable along respective axes that are not parallel to the lateral surfaces 51 and 53 of cases 50. Moving of focal zones 41 and 43 is in accordance with (a) movement of either one or both of the transducers defining elements 40 and/or 42 along the respective axes of first and second support structures 30 and 32 (e.g., while structures 30 and 32 remain in place with respect to the skin of the subject), and/or (b) movement of either one or both lenses 70 and 72 along the respective axes of first and second support structures 30 and 32 (e.g., while structures 30 and 32 remain in place with respect to the skin of the subject).

For some applications of the present invention, the transducer defining element 42 is replaced by a curved acoustic reflector which reflects and focuses the through-transmitted acoustic radiation in a pulse-echo mode from the transducer defining element 40 back toward the center of tissue portion 122 and even toward the transducer defining element 40. Typically, when the focal point of the acoustic reflector aligns with the focal zone of the transducer defining element 40, a maximal echo will be generated.

Typically, acoustic elements 40 and 42 of FIG. 3A function in both treatment and monitoring modes as described hereinabove with respect to acoustic elements 40 and 42, with reference to respective applications of FIG. 1. It is to be noted that for some applications, at least one of acoustic elements 40 and/or 42 functions to reflect through-transmitted and/or scattered energy, or is replaceable with an acoustic reflector.

For some applications of the present invention, cases 50 do not provide tracks 52, and lenses 70 and 72 are fixedly disposed with respect to case 50 and the non-focused transducers defining elements 40 and 42, respectively. In such an application, acoustic lenses 70 and 72 each typically comprise a respective flexible acoustic lens whose concavity is alterable. Typically, the curvature of the lens surface can be altered by a hydraulic, electromechanical, or a mechanical system. In such an application, the hydraulic or mechanical system applies pressure to lenses 70 and 72 in such a manner that fluid is extracted from (or introduced within) lenses 70 and 72. Such a pressure alters the curvature of respective concave interfaces 71 of lenses 70 and 72. Changing the concavity of lenses 70 and 72 changes the focal zones of lenses 70 and 72, thereby facilitating mechanical steering of the focal zones in order to achieve confocal transmission at the center of tissue portion 122. For some applications of the present invention, each lens 70 or 72 is fixed in place with respect to the housing by respective springs which provide force to the lenses to hold it in place and release the lenses when the springs are compressed.

Reference is now made to FIGS. 1-3A. It is to be noted that respective components of apparatus 20, 22, and 26 described herein may be used in various combinations in order to create a desired form of confocal acoustic transmission.

FIG. 3B shows apparatus 90 comprising: (a) a first acoustic lens 80 disposed in acoustic communication between acoustic element 40 and tissue portion 122, and (b) a second acoustic lens 82 disposed in acoustic communication between acoustic element 42 and tissue portion 122, in accordance with some applications of the present invention. Each lens 80 and 82 is shaped to define a respective lateral planar surface 81 disposed in acoustic communication with acoustic elements 40 and 42, respectively. Lenses 80 and 82 are each shaped to define two or more, e.g., a plurality of, concave interfaces 83, or surfaces, at respective lateral surfaces of lenses 80 and 82 that are in acoustic communication with tissue portion 122 and with lateral surfaces 51 and 53, respectively, of cases 50. Each concave interfaces 83 has a radius of curvature of between 0.5 mm and 5 mm, e.g., 1 mm. Typically, concave interfaces 83 of lenses 80 and 82 focus energy that is transmitted from acoustic elements 40 and 42, respectively, at the surface of skin 24 and respective focal zones, e.g., treatment areas. Typically, in response to the energy focused by interfaces 83 at the surface of the skin, respective small holes at the surface of skin 24 are generated having a diameter of around 0.1 mm (by way of illustration and not limitation) and a depth of 0.5 mm from the surface of skin 24 (by way of illustration and not limitation). In such an application, the transducers defining elements 40 and 42 typically transmit energy at a frequency of between 10 MHz and 20 MHz. Lenses 80 and 82 comprise a plastic, e.g., acrylic such as Perspex (Poly(methyl methacrylate) (PMMA)), by way of illustration and not limitation. For some applications of the present invention, lenses 80 and 82 are disposable and easily removable, by a user of the apparatus, from other components of the apparatus. Following each treatment, the lens may be replaced by another lens. In such an application, hygiene is maintained during application of the device to various subjects.

For some applications of the present invention, acoustic elements 40 and 42 each comprise at least one single non-focused transducer, e.g., an array of non-focused transducers. Alternatively, acoustic elements 40 and 42 each comprise at least one single focused transducer, e.g., an array of focused transducers. In either application, acoustic elements 40 and 42 transmit high frequency energy to the surface of skin 24 of tissue portion 122 that is drawn between first and second support structures 30 and 32. Each concave interface focuses treatment energy transmitted from acoustic elements 40 and/or 42 in order to create a plurality of ultrasound treatment areas, or focal zones 87, at the surface of skin 24 of portion tissue 122 in order to generate small holes. The holes: (a) facilitate transdermal delivery and uptake of applied drugs, solutions, and/or creams to the holes for aesthetic and medical purposes, (b) facilitate insertion of skin fillers such as collagen into the holes, and (c) induce the natural production of collagen in response to the body sensing “damage” to the surface of skin 24. The drugs, solutions, and/or creams applied to the skin effect (a) skin regeneration, (b) tightening of the skin, (c) fat destruction, and/or (d) any other effect of drugs, solutions and/or creams.

Creating the small holes in the skin will cause the skin to regenerate and tighten, since collagenous tissue will be naturally regenerated.

In order to move focal zones 87 with respect to tissue portion 122, each lens 80 and 82 slides axially (i.e., in the directions as indicated by arrows 54) along respective axes of structures 30 and 32 that are not-parallel, e.g., perpendicular to, lateral surfaces 51 and 53 of cases 50. Tracks 52 are disposed along the respective axes that are not-parallel, e.g., perpendicular, to lateral surfaces 51 and 53 of cases 50, and enable lenses 80 and 82 to slide along tracks 52 and along the axes that are not-parallel to lateral surfaces 51 and 53 of cases 50. Thus, lenses 80 and 82 are moveable with respect to tissue portion 122 and with respect to support structures 30 and 32, respectively. Typically, cases 50 comprise an acoustic coupling medium (e.g., water or oil) which maintains acoustic coupling between tissue portion 122, the transducers defining elements 40 and 42, and lenses 80 and 82 as they are axially moved along tracks 52 within cases 50.

Moving of the respective focal zones is in accordance with (a) movement of either one or both elements 40 and/or 42 along the respective axes of first and second support structures 30 and 32 (e.g., while structures 30 and 32 remain in place with respect to the skin of the subject), and/or (b) movement of either one or both lenses 80 and 82 along the respective axes of first and second support structures 30 and 32 (e.g., while structures 30 and 32 remain in place with respect to the skin of the subject).

It is to be noted that tracks 52 are shown by way of illustration and not limitation, and that the desired movement of lenses 80 and 82 may be accomplished without tracks, using any other suitable means known in the art. It is to be further noted that the transducers defining elements 40 and/or 42 may be moveable along the respective axes of structures 30 and 32 (described hereinabove) that are not parallel to the lateral surfaces 51 and 53 of cases 50.

For some applications of the present invention, acoustic elements 40 and/or 42 receive through-transmitted energy in order to monitor treatment of tissue, in respective manners as described hereinabove with reference to FIG. 1.

Reference is now made to FIG. 3B. It is to be noted that apparatus 90 is shown as comprising both first and second support structures 30 and 32 by way of illustration and not limitation. For example, some applications of the present invention include use of first support structure 30 independently of second support structure 32, acoustic element 42, and lens 82. In these applications, a portion of tissue is not pinched by the apparatus, but may or may not be pinched by an operating physician. Whether or not tissue is pinched, lateral surface 51 of first support structure is placed against a surface of the skin of the subject by a user of the apparatus, e.g., a physician or the subject, and acoustic element 40 (e.g., an ultrasound transducer) transmits the acoustic energy toward the surface of the skin of the subject. The energy transmitted from acoustic element 40 is focused via lens 80 to respective focal zones at the surface of the skin of the subject, thereby creating the holes, in a manner as described hereinabove.

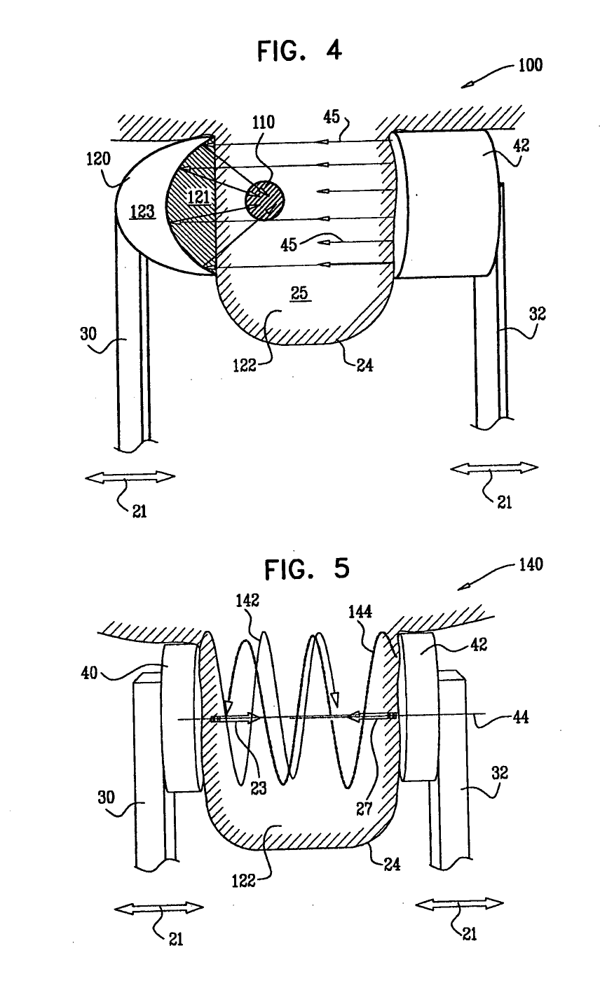

Reference is now made to FIG. 4, which is a schematic illustration of apparatus 100 comprising an acoustic element 42 coupled to support structure 32 and a shaped acoustic reflector 120 coupled to support structure 30, in accordance with some applications of the present invention. Reflector 120 defines an acoustic element. For some applications of the present invention, acoustic element 42 comprises at least a single, non-focused, flat ultrasound transducer, e.g., an array of non-focused ultrasound transducers which transmit acoustic beams 45, e.g., in a pulse/burst mode or in a continuous wave mode, toward tissue portion 122. Reflector 120 is disposed with respect to acoustic element 42 in a manner in which at least a portion of beams 45 transmitted from acoustic element 42 are reflected from acoustic reflector 120 toward a focal zone 110 which is typically in the center of tissue portion 122, e.g., equidistant from (a) an interface between reflector 120 and skin 24 of tissue portion 122, and (b) from an interface between acoustic element 42 and skin 24 of tissue portion 122. Typically, such reflection generates nonconfocal transmission of energy toward focal zone 110. For some applications of the present invention, waves are transmitted in a manner in which at least one focal zone is generated in the portion of skin and the underlying tissue substantially midway between acoustic element 42 and reflector 120. It is to be noted that substantially midway between element 42 and reflector 120 includes a majority of portion 122 which excludes the hatched portions which represents the stratum corneum.

Typically, acoustic reflector 120 is shaped to define a concave (e.g. parabolic or ellipsoidal) reflecting portion 123 coupled to a generally non-refracting coupling medium, comprising polyurethane, by way of illustration and not limitation. The concave surface faces portion 122. For some applications of the present invention, acoustic reflector 120 and/or acoustic element 42 may be replaced with a focused ultrasound transducer having a curved surface that faces and is in acoustic communication with the lateral surface of the support structure, which interfaces with tissue of portion 122, as described hereinabove with reference to FIG. 2. Once tissue portion 122 is clamped between acoustic element 42 and reflector 120 (i.e., by moving support structures 32 and 30, respectively in the directions as indicated by arrows 21), and transmission of energy from acoustic element 42 is initiated, neither acoustic element 42 nor reflector 120 is movable with respect to tissue portion 122 and with respect to support structures 30 and 32. Typically, since neither reflector 120 nor acoustic element 42 is moveable with respect to each other, focal zone 110 is spatially fixed relative to reflector 120.

For some applications of the present invention, acoustic element 42 comprises an annular phased array of ultrasound transducers which can steer (in a manner as described hereinabove with reference to FIG. 1) its focal zone electronically to align with the focal zone 110 of reflector 120.

Reference is now made to FIG. 5, which is a schematic illustration of apparatus 140 comprising acoustic elements 40 and 42 which are configured to generate constructive interference in tissue portion 122, in accordance with some applications of the present invention. In such an application, acoustic elements 40 and 42 each comprise a single non-focused flat, transducer (e.g., an array of non-focused flat, transducers) which is configured to generate a high intensity of ultrasound energy in tissue portion 122. Alternatively, elements 40 and/or 42 each comprise a focused transducer.

The ultrasound transducer defining element 40 transmits (in a direction as indicated by arrow 23) an acoustic wave 142 toward acoustic element 42, while ultrasound transducer 42 simultaneously transmits (in a direction as indicated by arrow 27) an acoustic wave 144 toward the ultrasound transducer defining element 40. (Wavelengths of the waves are not to scale in the figure.) The parameters, e.g., frequency and phase, of each transmitted wave 142 and 144 are set such that regions of constructive interference are obtained within tissue portion 122. In these regions, the intensity of the ultrasound transmission is high, and energy is accumulated. If the transmission duration is sufficiently long, damage is induced in the tissue. Since the distance between the two elements and the speed of sound of the tissue is known prior to transmission of waves 142 and 144 from the transducers defining elements 40 and 42, respectively, the position of these interference zones is precisely controllable and alterable by altering the parameters, e.g., the phases and frequencies, of transmitted waves 142 and 144.

Reference is now made to FIGS. 1-5. For some applications of the present invention, apparatus described herein may be used in combination with a tracking system comprising a plurality of reference sensors in order to assess the location of treated tissue by registering the relative spatial coordinates of the acoustic elements and/or anatomy of the subject. Techniques described herein may be used in combination with the tracking system described in the above-cited PCT Publications to Azhari.

Reference is again made to FIGS. 1-5. For some applications of the present invention, the acoustic elements described herein are configured to transmit ultrasound energy in a continuous wave (CW) mode, in which a relatively long train of a sinusoidal wave is transmitted. In such an application, heating and cell implosion are effected as a result of the treatment procedure. For this particular application, the acoustic elements transmit ultrasound energy at a high frequency range of about 1-5 MHz, e.g., 3 MHz) in the CW mode. Such transmission heats portion 122 of tissue 25 to a relatively-high temperature of about 40-70 C, e.g., 45 C. For some applications of the present invention, the temperature is evaluated using techniques described in PCT Publication WO 06/018837 to Azhari.

In some applications of the present invention, a continuous wave of alternating positive-pressure and negative-pressure pulses of imploding waves is applied to the treatment area. In response to the continuous wave, increased temperature is generated at the central location of the adipose cell. Such a combined effect of continuous negative and positive pressure is configured to destroy the cell at the central location.

For some applications of the present invention, the acoustic elements described herein are configured to transmit ultrasound energy in a burst mode, in which a sharp pulse is transmitted. For some applications of the present invention, the acoustic elements transmit ultrasound energy at a frequency of, typically but not necessarily, about 250 kHz. After reaching the desired temperature, high intensity implosion waves obtained using the burst mode are inwardly-directed to tissue portion 122. It is to be noted that bursts of pulses with positive amplitudes and/or negative amplitudes may be applied to tissue 25 of the subject. For some applications, the inwardly-directed wave creates a negative pressure pulsed wave. In such a case, a strong and rapid decrease in pressure at the focal point is created. As a result, tissue cells and/or connective tissue are subjected to tearing stresses causing irreversible damage thereto.

In some applications of the present invention, the acoustic elements generate a series of strong negative-pressure pulses of imploding waves which are directed toward the treatment area at a transmission rate of several kilohertz. Such strong negative-pressure pulses effect radial stretching and tearing of the adipose cell. In such an application, implosion, thermal ablation, as well as some localized cavitation may occur in the treatment area. In order to reduce the level of cavitation, a rapid series of high-frequency pulses, typically at a central frequency greater than 1 MHz, may be transmitted during a single treatment. For some applications of the present invention, a series of positive-pressure pulses of implosion waves are applied to the treatment area in alternation with negative-pressure pulses. The application of the positive-pressure pulses mitigates and counters the cavitation effect generated by the negative-pressure pulses. In such an application, a plurality of series of negative-pressure pulses are interspersed by series of positive-pressure pulses.

It is to be noted that applications of the present invention may be applied to treatments such as lipolysis and body contouring, face-localized molding of adipose tissue, mentoplasty and neck lift. Some applications of the present invention may be applied to treat lipomas. Some applications of the present invention may be applied to treat and minimize eye bags of the subject. For face, chin, and neck treatments, a small probe shaped to define a diameter of between about 2-4 cm is typically used. Other applications such as hair removal may be effected by using elements that create a linear focal zone in combination with some of the treatment procedures described hereinabove. For some applications, one element creates the linear focal zone. Alternatively, multiple elements are used to create several linear focal zones.

Some applications of the present invention may be applied to treat conditions of the face of the patient because the support elements of the housing of the apparatus pinch portions of the tissue of the subject and can localize treatment to superficial facial tissue.

It is noted that although some applications of the present invention are described with respect to the use of ultrasound, the scope of the present invention includes replacing the ultrasound transducers described herein with transducers of other forms of energy, such as electromagnetic radiation.

Some applications of the present invention described herein may be used, for example, for cosmetic purposes, such as by placing the apparatus described hereinabove with reference to FIGS. 1-5 in contact with skin of the subject and treating tissue. The scope of the present invention includes application of the techniques described herein to tissue other than skin, as well. For example, apparatus described hereinabove with reference to FIGS. 1-5 may be sized for placement during surgery on an intrabody organ of the subject, such as the heart or an abdominal organ.

It is to be noted that techniques described herein with reference to FIGS. 1-5 may be used in order to tighten the collagen matrix in the treated tissue. For example, the heat generated in the tissue in response to treatment energy applied by the acoustic elements is configured to tighten the collagen matrix in the portion of the skin and underlying tissue that is drawn between the first and second support structures. For some applications of the present invention, the heat generated as a result of the ultrasound transmission shortens the connective tissue (e.g., tendons) of the facial muscles which creates an effect similar to a facelift. For some applications of the present invention, radiofrequency energy is transmitted in combination with ultrasound in order to achieve skin therapy. In such an application, the radiofrequency energy is configured to treat tissue 25 of portion 122 that is closer to skin 24, while the ultrasound energy transmitted from the acoustic elements reaches deep within tissue portion 122, e.g., to the center of portion tissue 122, as described hereinabove.

For some applications, a chemical composition that destroys adipose tissue (e.g., Kythera) is injected into the surface of the skin prior to application of the ultrasound energy by the device. The heat generated by the subsequent application of the ultrasound energy to the tissue then enhances the effect of the injected substance. Typically, the heat generated as a response of the transmitted ultrasound energy can be focused and localized. Thus, for applications in which ultrasound energy is used in combination with the chemical composition, the heat generated from the ultrasound transmission helps focus and localize the effects of the chemical composition, thereby facilitating a more controlled treatment of tissue. For some applications, the device described herein may be used in combination with Botox injections.

For some applications of the present invention, the devices, treatments, and monitoring techniques described herein are used in order to treat varicose veins. The transducers effecting treatment of varicose veins (and other treatments described herein) operate in a closed loop manner in which the monitoring of the treatment will automatically effect a change in parameters (e.g., energy intensity or duration of pulses) of the therapy/treatment mode in the absence of intervention by an operator.

It is to be noted that the scope of the present invention includes transmitting energy to slightly beneath the plane defined by the housing, i.e., lower surfaces 55 and 57 of respective cases 50 of structures 30 and 32. Energy is transmitted, by way of illustration and not limitation, up to 5 or up to 10 degrees beneath the plane defined by the housing, in order to treat tissue in the vicinity of the housing.

Treatments using the treatment system may include, as appropriate, causing heating, tissue damage, thermal ablation, acoustic streaming, mechanical irritation, cell structure alteration, augmented diffusion, and/or a cavitation effect. Typically, the treatment system comprises circuitry for configuring the applied energy as high intensity focused ultrasound (HIFU), using techniques known in the art.

For some applications of the present invention, the monitoring system generally continuously generates acoustic maps or images, depicting changes occurring during a treatment of the tissue within the housing. For some applications, this allows an operator of the treatment system to monitor the progress of a treatment, and to alter a parameter of the treatment in response thereto. Such a parameter may include, for example, a location of a focus of the HIFU, a positioning of the housing on the subject's skin, or a strength of the applied energy. Alternatively or additionally, the treatment system and monitoring system operate in a closed loop fashion, whereby an output of the monitoring system (e.g., a location of fatty tissue) is used as an input parameter to the treatment system, such that the treatment system can adjust its operating parameters in response to the output of the monitoring system (and, for example, heat the fatty tissue).

For some applications of the present invention, the apparatus comprises a tracking system comprising reference sensors configured to track progress of treatments conducted on different days, or during the same procedure, by registering and recording the spatial location of the treated tissue. Typically, the spatial localization is achieved in comparison to corresponding predefined anatomical locations of the subject with respect to the housing. Alternatively, the spatial localization corresponds to coordinates in a room with respect to the housing.

It is to be further noted that, for some applications, the housing described herein coupled to the first and second support structures is flexible, e.g., to allow the treatment of limbs or other curved body parts. Alternatively, the housing is generally rigid. For some applications, the housing comprises a flexible cuff configured to surround a limb of the subject designated for treatment. The subsets of acoustic elements are typically arranged around the cuff on a circle defined by the cuff. For some applications, the acoustic elements are configured to remain fixed at their respective locations with respect to the cuff, while the cuff moves about the limb. For other applications, an electromechanical system moves at least a portion of the acoustic elements to different locations on the cuff.

Reference is now made to FIGS. 1-5. For some applications, the apparatus does not comprise a housing; rather, the first and second support structures comprise hand-held devices resembling wands, which are placed by the user, e.g., a physician or the subject, at two different portions of the skin of the subject. In such an application, the distance between the first and second support structure is controlled by the user of the apparatus. It is to be further noted that components and applications of devices as shown and described may be interchangeable between the components and applications.

Techniques and apparatus described herein may be practiced in combination with techniques and apparatus described in one or more of the following:
U.S. Provisional Patent Application 60/780,772 to Azhari et al., entitled, “A method and system for lypolysis and body contouring,” filed Mar. 9, 2006;
U.S. Provisional Patent Application 60/809,577 to Azhari et al., entitled, “A device for ultrasound monitored tissue treatment,” filed May 30, 2006;
U.S. Provisional Patent Application 60/860,635 to Azhari et al., entitled, “Cosmetic tissue treatment using ultrasound,” filed Nov. 22, 2006;
U.S. patent application Ser. No. 11/651,198 to Azhari et al., entitled, “A device for ultrasound monitored tissue treatment,” filed Jan. 8, 2007;
U.S. patent application Ser. No. 11/653,115 to Azhari et al., entitled, “A method and system for lipolysis and body contouring,” filed Jan. 12, 2007, which is a continuation-in-part of U.S. patent application Ser. No. 11/651,198, to Azhari et al., entitled, “A device for ultrasound monitored tissue treatment,” filed Jan. 8, 2007;
PCT Patent Application Publication WO 07/102,161 to Azhari et al., entitled, “A device for ultrasound monitored tissue treatment,” filed on Mar. 8, 2007;
U.S. Provisional Patent Application 60/999,139 to Azhari et al., entitled, “Implosion techniques for ultrasound,” filed on Oct. 15, 2007;
U.S. Provisional Patent Application 61/096,419 to Azhari et al., entitled, “A device for ultrasound treatment and monitoring tissue treatment,” filed Sep. 12, 2008;
PCT Patent Application Publication PCT/IL2008/001390 to Azhari et al., entitled, “Implosion techniques for ultrasound,” filed on Oct. 22, 2008; and/or
U.S. Provisional Patent Application 61/096,516 to Azhari, entitled, “Virtual ultrasonic scissors—A non-invasive method for tissue treatment,” filed Sep. 12, 2008, and to a PCT application to Azhari, entitled “Virtual ultrasonic scissors,” filed on even date herewith that claims priority of the '516 Azhari provisional application.

All of these applications are incorporated herein by reference.

For some applications, techniques described herein are practiced in combination with techniques described in one or more of the references cited in the Background section of the present patent application, which are incorporated herein by reference.

It is noted that although some applications of the present invention are described with respect to the use of ultrasound, the scope of the present invention includes replacing the ultrasound transducers described herein with transducers of other forms of energy, such as electromagnetic radiation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a first support structure having a lower surface and a lateral surface, the first support structure being configured for placement on a first location on skin of a subject;
a second support structure having a lower surface and a lateral surface, the second support structure being configured for placement on a second location on the skin of the subject, at least one of the first and second support structures being moveable with respect to the other so as to draw a portion of the skin and underlying tissue of the subject between the respective lateral surfaces of the first and second support structures;
at least one ultrasound transducer moveably coupled to the first support structure, the ultrasound transducer being moveable along an axis of the first support structure that is not parallel to the lateral surface of the first support structure, and configured to transmit toward the portion of skin and underlying tissue one or more forms of acoustic radiation energy, at least one of the one or more forms of energy including treatment energy; and
at least one acoustic element coupled to the second support structure.

2. The apparatus according to claim 1, wherein the axis is perpendicular to the lateral surface of the first support structure, and wherein the ultrasound transducer is moveable along the axis that is perpendicular to the lateral surface of the first support structure.

3. The apparatus according to claim 1, wherein the ultrasound transducer and the acoustic element are arranged such that transmission of energy from the ultrasound transducer generates a focal zone which moves within the portion of skin and underlying tissue in accordance with movement of the at least one ultrasound transducer.

4. (canceled)

5. The apparatus according to claim 1, wherein the apparatus is configured to configure the treatment energy to generate a standing wave in the portion of skin and underlying tissue.

6-7. (canceled)

8. The apparatus according to claim 1, wherein:
at least a portion of the lateral surface of the first support structure is configured to be disposed at a first interface between the portion of skin and the housing,
at least a portion of the lateral surface of the second support structure is configured to be disposed at a second interface between the portion of skin and the housing, the second interface being opposite the first interface, and
the ultrasound transducer and the acoustic element are configured to facilitate confocal transmission of the one or more forms of energy in the portion of skin and underlying tissue, from the opposing interfaces.

9-11. (canceled)

12. The apparatus according to claim 1, further comprising a processing unit, wherein the processing unit is configured to detect a position change of the at least one of the support structures in response to movement of the at least one of the support structures, and responsively thereto, to facilitate monitoring of the parameter of the tissue underlying the skin.

13-19. (canceled)

20. The apparatus according to claim 1, wherein the first ultrasound transducer is configured to transmit the energy in a manner in which the first focal zone is generated in the portion of skin and the underlying tissue substantially midway between the first ultrasound transducer and the acoustic element.

21. The apparatus according to claim 1, wherein the acoustic element comprises an acoustic reflector configured to reflect the energy transmitted from the first ultrasound transducer toward at least the first focal zone.

22-24. (canceled)

25. The apparatus according to claim 1, wherein:
the apparatus further comprises a first acoustic lens configured to be disposed between the ultrasound transducer and the portion of skin and the underlying tissue within the at least a part of the housing, and
the first lens is moveable with respect to the first support structure along the axis of the first support structure that is not parallel to the lateral surface of the first support structure.

26. The apparatus according to claim 25, wherein the first lens is configured to alter a position of the first focal zone by being moved along the axis of the first support structure.

27-31. (canceled)

32. The apparatus according to claim 25,
wherein the acoustic lens is shaped to provide a first lateral surface and a second lateral surface having two or more concave surfaces, and wherein the acoustic lens is configured to be disposed between the ultrasound transducer and a first surface of the portion of skin and underlying tissue in a manner in which:
the first lateral surface of the acoustic lens is in acoustic communication with the ultrasound transducer, and
the second lateral surface of the acoustic lens is in acoustic communication with the lateral surface of the first support structure and with the first surface of the portion of skin and underlying tissue, and
wherein each one of the two or more concave surfaces of the second lateral surface of the acoustic lens is configured to focus the energy toward a respective focal zone in the first surface of the portion of skin and underlying tissue.

33. The apparatus according to claim 32, wherein each one of the two or more concave surfaces of the second lateral surface of the acoustic lens is configured to facilitate generating of a respective hole at each focal zone in the first surface of the portion of skin and underlying tissue.

34. The apparatus according to claim 25, wherein:
the apparatus further comprises a second acoustic lens configured to be disposed between the second acoustic element and the portion of skin and the underlying tissue within the at least a part of the housing,
the second lens is moveable with respect to the second support structure along an axis of the second support structure that is not parallel to the lateral surface of the second support structure, and
the second lens is configured to focus the transmitted energy to at least one second focal zone.

35-38. (canceled)

39. The apparatus according to claim 1, wherein the second support structure is configured to be disposed with respect to the ultrasound transducer such that the one or more forms of energy are through-transmitted toward the acoustic element, and wherein at least a portion of the through-transmitted energy is received at least one component selected from the group consisting of: the at least one ultrasound transducer and the at least one acoustic element.

40. (canceled)

41. The apparatus according to claim 39, further comprising a processing unit configured to monitor a change in a parameter of the tissue underlying the skin, responsively to the received energy.

42. (canceled)

43. The apparatus according to claim 41, wherein:
the acoustic element comprises an ultrasound reflector configured to reflect the through-transmitted energy transmitted from the ultrasound transducer,
the ultrasound transducer is configured to receive at least a portion of the reflected energy, and
the processing unit is configured to monitor a change in the parameter of the tissue responsively to the reflected energy.

44-49. (canceled)

50. A method, comprising:
placing at a first location on skin of a subject at least one ultrasound transducer moveably coupled to a first support structure, the ultrasound transducer being configured to transmit through the skin one or more forms of acoustic radiation, at least one of the one or more forms of energy including treatment energy,
placing at a second location on the skin of the subject at least one acoustic element moveably coupled to a second support structure;
drawing at least a portion of the skin and underlying tissue between the at least one ultrasound transducer and the at least one acoustic element;
moving the at least one ultrasound transducer with respect to the first support structure along an axis that is not parallel to a lateral surface of the first support structure;
transmitting from the at least one ultrasound transducer one or more forms of energy through the portion of skin and the underlying tissue; and
generating confocal transmission responsively to the transmitting.

51-54. (canceled)

55. The method according to claim 50, wherein transmitting the energy comprises generating constructive interference in the portion of skin and underlying tissue.

56-61. (canceled)

62. The method according to claim 50, wherein placing the acoustic element comprises placing the acoustic element with respect to the ultrasound transducer such that the one or more forms of energy are through-transmitted toward the acoustic element.

63. (canceled)

64. A method, comprising:
placing in communication with a surface of a portion of skin of a subject:
at least one acoustic element, and
at least one acoustic lens between the acoustic element and the surface of the portion of the skin of the subject, the acoustic lens having a first lateral surface that is disposed in acoustic communication with the acoustic element, and a second lateral surface having two or more concave interfaces in acoustic communication with the surface of the portion of the skin of the subject;
transmitting from the at least one acoustic element one or more forms of acoustic radiation energy, at least one of the one or more forms of energy including treatment energy, toward the surface of the portion of the skin of the subject;
focusing the energy through each of the two or more concave interfaces to a respective focal zone at the surface of the portion of the skin of the subject; and
responsively to the focusing, generating two or more treatment areas in the surface of the portion of the skin of the subject.

65-67. (canceled)

68. The method according to claim 64, further comprising, in conjunction with the generating the two or more holes in the surface of the portion of the skin of the subject, facilitating uptake by the surface of the portion of the skin of the patient one or more substances selected from the group consisting of: a medication, a solution, or a cream, by applying the one or more selected substances to the surface of the portion of the skin of the subject.

69-89. (canceled)
